(19) United States
(12) Patent Application Publication
(10) Pub. No.: US 2015/0216998 A1
(43) Pub. Date: Aug. 6, 2015

(54) ENDO180-TARGETED PARTICLES FOR SELECTIVE DELIVERY OF THERAPEUTIC AND DIAGNOSTIC AGENTS

(71) Applicants: QBI ENTERPRISES LTD., NES Ziona (IL); RAMOT AT TEL-AVIV UNIVERSITY LTD., Tel Aviv (IL)

(72) Inventors: Elena Feinstein, Rehovot (IL); Dan Peer, Kiryat Ono (IL)

(73) Assignees: RAMOT AT TEL-AVIV UNIVERSITY LTD., Tel Aviv (IL); QBI ENTERPRISES LTD., Nes Ziona (IL)

(21) Appl. No.: 14/367,922

(22) PCT Filed: Dec. 31, 2012
(86) PCT No.: PCT/IL2012/000405

§ 371 Date: Jun. 22, 2014

Related U.S. Application Data
(60) Provisional application No. 61/582,373, filed on Jan. 1, 2012.

(51) Int. Cl. A61K 47/48; A61K 31/713; A61K 31/704

(52) U.S. Cl. CPC A61K 47/48823; A61K 31/704; A61K 31/713; A61K 47/48561

(57) ABSTRACT

Disclosed herein are compositions comprising lipid based particles and anti-ENDO180 antibodies and to methods of using the same for targeted delivery of therapeutic agents to cancer and fibrotic cells useful for treating cell proliferative diseases or disorders including fibrosis, cancer and to attenuate tumor progression.

### RELATED APPLICATIONS

This application claims the benefit of U.S. Provisional Application Ser. No. 61/582,373 filed Jan. 1, 2012 entitled “ENDO180-Targeted Particles for Selective Delivery of Therapeutic and Diagnostic Agents” and incorporated herein by reference in its entirety and for all purposes.

### SEQUENCE LISTING

This application incorporates-by-reference nucleotide and/or amino acid sequences which are present in the file named “230-PCT1_SEQLISTING.ST25.txt”, which is 33 kilobytes in size, and which was created Dec. 31, 2012 in the IBM-PCT machine format, having an operating system compatibility with MS-Windows.

### FIELD OF THE INVENTION

Disclosed herein are compositions comprising carrier moieties (such as lipid based particles), and anti-ENDO180 targeting moieties (such as anti-ENDO180 antibodies) and to methods of using the same for delivery of therapeutic and/or diagnostic agents to cells and tissue expressing ENDO180, including tumor cells, macrophages, endothelial cells and fibrotic cells. The compositions and methods are useful for treating cell proliferative diseases, or disorders including fibrosis, cancer, or inflammation, and for controlling (modulating) tumor progression.

### BACKGROUND OF THE INVENTION

The ENDO180 Receptor, also known as CD280, uPARAP (urokinase plasminogen activator receptor associated protein) and mannose receptor C type 2 (MRC2), is a recycling endocytic receptor that directs bound ligands to degradation in the endosomes. It is part of a triple complex with urokinase type plasmin activator (uPA) and urokinase-type plasmin activator receptor (uPAR), and is involved in the production of plasmin from plasminogen. Plasmin, in turn, is known to play a role in both extracellular matrix (ECM) turnover and proteolytic conversion of latent TGF-beta into its active form.

ENDO180 shares homology with the macrophage mannose receptor family: mannose receptor, phospholipase A2 and DEC-205/MR6 (Isacke et al., 1990 Mol. Cell. Biol. 10:2606-2618; Sheikh et al., 2000, J. Cell. Sci. 113: 1021-1032; Behrendt et al., 2000, J. Biol. Chem. 275: 1993-2002). ENDO180 is unusual in the family of mannose receptors in that it is targeted from the plasma membrane to the recycling endosomes rather than to a late endosome/lysosome compartment (Howard and Isacke, 2002. JBC 35:32320-31) and functions in cell motility and remodeling of the extracellular matrix by promoting cell migration and uptake of collagens for intracellular degradation (Behrendt. 2004 Biol Chem. 385(2):103-36; Kjoller et al, 2004 Exp Cell Res. 293(1):106-16; Wienke et al., 2007 Cancer Res. 67(21): 10230-40).

PCT Patent Application Publication No. WO 2004/100759 relates to methods of diagnosing and treating, respectively, diseases associated with ENDO180. PCT Patent Application Publication No. WO 2010/111198 provides anti-ENDO180 antibodies, compositions comprising same and uses thereof.

### Lipid Complexes

US 2009/0232730 discloses a method for producing immunoliposomes. US 2010/0008937 discloses leukocyte selective delivery agents.

A targeted system for delivery of therapeutic and diagnostic agents would be of great value.

### SUMMARY OF THE INVENTION

Disclosed herein are compositions for selective and targeted delivery of therapeutic and/or diagnostic agents to aberrantly proliferating cells. The compositions comprise ENDO180-targeting moieties and carrier moieties, further comprising a therapeutic and/or diagnostic agent for targeted delivery of the therapeutic or diagnostic agent to a cell expressing an ENDO180 receptor. The composition is useful for targeted delivery of at least one diagnostic agent and/or therapeutic agent including a small molecule, such as an oligonucleotide, an antibody or fragment thereof, a polypeptide or peptide, or a combination thereof, to the intracellular space of a cell expressing the ENDO180 receptor. Without wishing to be bound to theory, the ENDO180 receptor is an endocytic receptor specifically expressed on activated myoblasts in fibrotic tissues and tumors and on subsets of tumor cells, on macrophages and on endothelial cells.

In one aspect disclosed herein is a composition comprising a) a carrier moiety; b) an ENDO180 targeting moiety; and c) an effective amount of a therapeutic agent and/or or a diagnostic agent.

In some embodiments the carrier moiety comprises a lipid based carrier, preferably a lipid particle (also referred to as a lipid-based nanoparticle). In some embodiments the carrier moiety and the targeting moiety are covalently bound or non-covalently associated. In preferred embodiments the carrier moiety comprises a lipid particle covalently bound to the targeting moiety. In some embodiments the lipid particle and the targeting moiety are covalently bound via a surface modification of the liposome with a synthetic polymer, a natural polymer or a semi synthetic polymer (comprising natural and synthetic elements). In some embodiments the synthetic polymer comprises a PEG moiety. In some embodiments the PEG moiety comprises NHS-PEG-DSPE [3-(N-succinimidyloxyglutaryl)aminopropyl, polyethyleneglycol-carbamyl distearoylphosphatidyl-ethanolamine]. In some embodiments the natural polymer comprises a saccharide including a polysaccharide and/or a glycosaminoglycan. In some embodiments the glycosaminoglycan comprises hyaluronic acid.

The polymer may be incorporated into the liposomal composition ab initio or may be combined with the prepared lipid particle.

In some embodiments the ENDO180 targeting moiety comprises an ENDO180 binding protein that binds an extracellular domain of an ENDO180 polypeptide present on a call and is internalized into the cell by the ENDO180 polypeptide. In some embodiments the ENDO180 polypeptide is substantially identical to an amino acid sequence set forth in SEQ ID NO:2, encoded by a polynucleotide substantially identical to a nucleic acid sequence set forth in SEQ ID NO:1. In some embodiments, the ENDO180 binding protein comprises an ENDO180 antibody or a functional fragment thereof capable of binding ENDO180.

In some embodiments the ENDO180 targeting agent is selected from
a. an isolated monoclonal antibody or an antigen-binding fragment thereof, produced by the hybridoma cell line E3-8D8 deposited with the BCCM under Accession Number LMBP 7203CB;
b. an antibody or an antigen-binding fragment thereof that binds to the same epitope as the antibody of (a);
c. a humanized version of the antibody or an antigen-binding fragment thereof of (a), or a humanized version of the antibody or antigen-binding fragment of (b);
d. a chimeric version of the antibody or an antigen-binding fragment thereof of (a), or a chimeric version of the antibody or antigen-binding fragment of (b);
e. a recombinant polypeptide or antigen-binding fragment thereof comprising the antigen binding domain of the antibody of (a) which is internalized in to a cell by the ENDO180 receptor;
f. an antigen-binding fragment of an antibody comprising a polypeptide substantially similar to SEQ ID NO: 6; and
g. a recombinant polypeptide comprising CDRs having an amino acid sequence substantially similar to amino acid sequences set forth in SEQ ID NO:7 and 8.

In some embodiments the antibody or fragment thereof is humanized or a chimeric antibody or fragment thereof.

The E3-8D8 monoclonal antibody is also known as 8D8, e3b3 and 8D8E3B3. In preferred embodiments the monoclonal antibody or the antigen-binding fragment thereof; the humanized version of the antibody or the antigen-binding fragment thereof; or the chimeric version of the antibody or the antigen-binding fragment thereof of the monoclonal antibody binds to ENDO180 on the surface of a cell and is internalized into the cell.

In some embodiments the ENDO180 antibody is selected from the group consisting of a full IgG, a monoclonal antibody, a polyclonal antibody, a human antibody, a humanized antibody, a Fab fragment, a Fab′ fragment, an F(ab′)2 fragment, the variable portion of the heavy and/or light chains thereof, a Fab miniantibody (MB), and a scFv, or a combination thereof. In some embodiments the ENDO180 antibody is an antibody or a fragment thereof that binds to the same epitope as the monoclonal antibody produced by the hybridoma cell line E3-8D8 deposited with BCCM under Accession Number LMBP 7203CB; in some embodiments the ENDO180 antibody is a humanized version of the antibody of the monoclonal antibody produced by the hybridoma cell line E3-8D8 deposited with BCCM under Accession Number LMBP 7203CB or a humanized antibody or fragment thereof. In some embodiments the ENDO180 antibody is a recombinant polypeptide comprising an antigen binding domain comprising an amino acid sequence set forth in SEQ ID NO:7 or a variant thereof which retains the ability to specifically bind ENDO180. In some embodiments the ENDO180 antibody is a recombinant polypeptide comprising a CDR, such as a heavy chain CDR3 domain, having an amino acid sequence substantially similar to an amino acid sequence set forth in SEQ ID NO:7 or a variant thereof; comprising one or more conservative amino acid substitutions. In some embodiments, the variant retains the ability to specifically bind ENDO180. In some embodiments the antibody further comprises a CDR, such as a light chain CDR3 domain having an amino acid sequence substantially similar to an amino acid sequence set forth in SEQ ID NO:8 or a variant thereof. In some embodiments, the variant retains the ability to specifically bind ENDO180.

In some embodiments the ENDO180 targeting moiety comprises a scFv recombinant polypeptide comprising an antigen-binding domain of the monoclonal antibody produced by the hybridoma cell line E3-8D8 (BCCM Accession Number LMBP 7203CB).

In some embodiments the ENDO180 targeting moiety comprises a scFv recombinant polypeptide comprising an amino acid sequence set forth in SEQ ID NO:6 (minibody, MB) or a variant thereof, which retains the ability to specifically bind ENDO180. In specific embodiments the antibody exhibiting binding affinity to ENDO180 receptor and comprising CDR3 domains set forth in SEQ ID NOS:7 and 8 is internalized by the receptor into the cell expressing ENDO180 upon contact of the antibody to the receptor.

In some embodiments the lipid particle comprises phophosphatidylcholine or a derivative thereof, phosphatidylglycerol or derivative thereof, or phosphatidylethanolamine or a derivative thereof, or a combination thereof. In some embodiments the lipid particle comprises one or more of distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC), dimyristoylphosphatidylcholine (DMPC), dioleoyl phosphatidylethanolamine (DOPE), dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), dipalmitoylphosphatidic acid (DPPA). In some embodiments the lipid particle comprises a cationic lipid, such as one or more of a cationic lipid selected from DOTMA and DOTP, or a combination thereof.

In some embodiments the lipid particle comprises one or more of hydrogenated soy phosphatidylcholine (HSPC), soy phosphatidylcholine (soy PC), dioleoyl phosphatidylethanolamine (DOPE). In some embodiments the lipid particle comprises dioleoyl phosphatidylethanolamine (DOPE). In some embodiments the lipid particle comprises 1,2-Bis(diphenylphosphino)ethane (DPPE). In some embodiments the lipid particle further comprises cholesterol. In some embodiments, the lipid particle further comprises soy PC.

In some embodiments the lipid particle comprises DOPE and cholesterol.

In one embodiment the lipid particle comprises HSPC, cholesterol and DOPE. In other embodiments the lipid particle comprises DOPE, cholesterol and DOTMA. In other embodiments, the lipid particle comprises HSPC, cholesterol, DOPE and DOTMA.

In some preferred embodiments lipid particle comprises Dioleoyl Phosphatidylethanolamine (DOPE), 1,2-di-O-octadecenyl-3-trimethylammonium propane (DOTMA) and cholesterol (Chol) at a molar ratio of about 4:2:1 (DOPE:DOTMA:Chol).

In other preferred embodiments the lipid particle comprises DOPE, Hydrogenated soybean phosphatidylcholine (HSPC), cholesterol and NHS-PEG-DSPE at a molar ratio of about 4.5:20: 75:0.5 (DOPE:HSPC:Chol: NHS-PEG-DSPE). In some embodiments, the lipid particle further comprises DOTMA.

In other preferred embodiments the lipid particle comprises soy PC, 1,2-Bis(diphenylphosphino)ethane (DPPE) and cholesterol at a molar ratio of about 3:1:1 (soy PC:DPPE:cholesterol).

In some embodiments, the lipid particle is about 85 to about 300 nm in diameter, preferably under 200 nm, such as about 85 nm to about 150 nm in diameter.

In some embodiments, the lipid particle comprises a zeta potential of about (−7) to about (−60), preferably about (−7) to about (−40), preferably about (−7) to about (−18).

In some embodiments the composition further comprises a moiety including at least one of a diagnostic agent and/or a therapeutic agent. In some embodiments the diagnostic agent comprises a detectable label, such as an imaging agent selected from the group consisting of a radioisotope, a fluorophore, a luminescent agent, a magnetic label, and an enzymatic label.

In some embodiments the therapeutic agent comprises one or more of a chemotherapeutic, a nucleic acid, a peptide, a polypeptide or a peptidomimetic, and antibody of functional fragment thereof. In some embodiments the chemotherapeutic is a small molecule. In some embodiments the small molecule is doxorubicin or mitomycin.

In some embodiments the therapeutic agent is selected from a nucleic acid and a non-nucleic acid.

In some embodiments, the non-nucleic acid compound is selected from the group consisting of a small molecule, a peptide, a polypeptide, a peptidomimetic, a glycolipid, and an antibody, or a combination thereof.

In some embodiments the therapeutic agent is a nucleic acid selected from an antisense compound, a chemically modified dsRNA compound, an unmodified dsRNA compound, a chemically modified siRNA compound, an unmodified siRNA compound, a chemically modified shRNA compound, an unmodified shRNA compound, a chemically modified miRNA compound, and an unmodified miRNA compound, a ribozyme, or combinations thereof. In various preferred embodiments the therapeutic agent is chemically modified siRNA. In some preferred embodiments, the therapeutic agent is an unmodified siRNA compound.

In some preferred embodiments the lipid particle comprises Dioleoyl Phosphatidylethanolamine (DOPE), 1,2-di-O-octadecenyl-3-trimethylammonium propane (DOTMA) and cholesterol (Chol), hyaluronic acid, an anti-ENDO180 antibody or an antigen-binding fragment of a humanized or chimeric anti-ENDO180 antibody and a therapeutic agent selected from doxorubicin, mitomycin C and a therapeutic nucleic acid molecule. In some embodiments the Dioleoyl Phosphatidylethanolamine (DOPE), 1,2-di-O-octadecenyl-3-trimethylammonium propane (DOTMA) and cholesterol (Chol) are present at a molar ratio of about 4:2:1 (DOPE:DOTMA:Chol)

In other preferred embodiments the lipid particle comprises DOPE, Hydrogenated soybean phosphatidylcholine (HSPC), cholesterol and NHS-PEG-DSPE, an anti-ENDO180 antibody or an antigen-binding fragment of a humanized or chimeric anti-ENDO180 antibody and a therapeutic agent selected from doxorubicin, mitomycin C and a therapeutic nucleic acid molecule. In some embodiments, the lipid particle further comprises DOTMA. In some embodiments, the DOPE, Hydrogenated soybean phosphatidylcholine (HSPC), cholesterol and NHS-PEG-DSPE are present at a molar ratio of about 4.5:20: 75:0.5 (DOPE:HSPC:Chol: NHS-PEG-DSPE).

In other preferred embodiments the lipid particle comprises soy PC, 1,2-Bis(diphenylphosphino)ethane (DPPE) and cholesterol, hyaluronic acid, an anti-ENDO180 antibody or an antigen-binding fragment of a humanized or chimeric anti-ENDO180 antibody and a therapeutic agent selected from doxorubicin, mitomycin C and a therapeutic nucleic acid molecule. In some embodiments the soy PC, 1,2-Bis(diphenylphosphino)ethane (DPPE) and cholesterol are present at a molar ratio of about 3:1:1 (soy PC:DPPE:cholesterol).

In another aspect, provided herein is a method of treating a subject afflicted with a proliferative disorder comprising administering to the subject a therapeutically effective amount of a composition comprising a) a carrier moiety; b) an ENDO180 targeting moiety and c) a therapeutic agent.

In another aspect, provided herein is a composition comprising a) a carrier moiety; b) an ENDO180 targeting moiety and c) a therapeutic agent, for use in therapy.

In another aspect provided herein is a composition comprising a) a carrier moiety; b) an ENDO180 targeting moiety and c) a therapeutic agent, for use in treating a proliferative disorder.

In some embodiments, the composition is administered systemically.

In some embodiments the proliferative disorder is selected from a solid tumor, a hematopoietic tumor, metastases, fibrosis and a macrophage associated disorder. In some embodiments, the proliferative disorder is a solid tumor or a hematopoietic tumor.

In some embodiments the tumor is an ovarian tumor, a breast tumor, osteoblastic/osteocytic cancer, prostate cancer, head and neck cancer, leukemia, renal cell carcinoma, or transitional cell carcinoma.

In some embodiments the fibrosis is liver fibrosis, myelofibrosis, kidney fibrosis for any reason (CKD including end-stage renal disease, ESRD); lung fibrosis (including interstitial lung fibrosis ILF); abnormal scarring (keloids) associated with all possible types of skin injury accidental and jatrogenic (operations); scleroderma; cardiofibrosis, failure of glaucoma filtering operation; intestinal adhesions.

In some embodiments the macrophage-associated disorder is inflammation or atherosclerosis.

Non-limiting examples of diseases and disorders include:
1. soft tissue sarcomas in which ENDO180 is expressed in the tumor and tumor stroma cells (activated myofibroblasts, neovasculature and infiltrating cells of macrophage-monocyte lineage);
2. carcinomas in which ENDO180 is expressed in the tumor stroma cells (activated myofibroblasts, neovasculature and infiltrating cells of macrophage-monocyte lineage);
3. carcinomas that express ENDO180 and have undergone epithelial-mesenchymal transition thus acquiring high metastatic potential;
4. leukemia expressing ENDO180 for example, from macrophage-monocyte lineage;
5. fibrotic diseases, for example of kidney, lung and liver with activated myofibroblasts;
6. diseases and disorders associated with macrophage including atherosclerosis and chronic inflammation.

In another aspect, provided herein is a method of diagnosing a proliferative disorder in a subject, comprising contacting a biological sample from the subject with a composition comprising a) a carrier moiety; b) an ENDO180 targeting moiety and c) a diagnostic agent; and comparing the level of diagnostic agent in the biological sample with that of a reference sample, such as a biological sample from a healthy subject.

In some embodiments, the biological sample for diagnosis may be taken from a bodily fluid or from a tissue. In some embodiments, the bodily fluid is selected from the group of fluids consisting of blood, lymph fluid, ascites, serous fluid, pleural effusion, sputum, cerebrospinal fluid, lacrimal fluid, synovial fluid, saliva, stool, sperm, blood and urine.

The present invention is explained in greater detail in the figures, description and claims hereinbelow.

### BRIEF DESCRIPTION OF THE FIGURES

FIG. 1 provides a schematic illustration of the process of generating targeted nanoparticles for nucleic acid (NA) molecule delivery.

FIGS. 2A and 2B show flow cytometry analysis with NRK-ENDO180 (2A), A549 (2B) cell lines incubated with 1 μg/ml Anti ENDO180 mAbs; Clone 8D8, clone 10C12, Minibody and a secondary Ab FITC goat anti-mouse (1.5 μg/ml). The peaks showing cells bound to anti-ENDO180 are labeled for clarity.

FIGS. 3A and 3B show flow cytometry analysis with LLC ENDO180 (3A), DU145 ENDO180 (3B) cell lines incubated with 1 μg/ml Anti ENDO180 mAbs; Clone 8D8, clone 10C12 and Minibody and a secondary Ab FITC goat anti-mouse (1.5 μg/ml). The peaks showing cells bound to anti-ENDO180 are labeled for clarity.

FIGS. 4A-4D show flow cytometry analysis with DU145 ENDO180 (4A), DU145 naive (4B), NRK ENDO180 (stably transfected) (4C) and A549 (4D) cell lines incubated with 1 μg/ml Anti ENDO180 mAb; Clone 8D8 (orange line, right most peak in all graphs) and Minibody (new batch, blue line, center peak in all graphs) both were labeled with Alexa fluor-647, in a comparison with control unstained cells (red line, left most peak in all graphs).

FIGS. 5A-5D shows cells which have internalized ENDO180 mAbs: 8D8 mAb into NRK-ENDO180 (5A) Minibody new batch into A549 cell line (5B) and 8D8 mAb into A549 (5C & 5D) using confocal microscope. Incubation time 1 hour at 37° C. with Alexa 488 labeled mAbs (red, left peak), (5.0 μg/ml each), Hoechst (azure, H 33342) 1:10,000, Cell Tracker™ (green, DilC18(5)-DS 1:5000). Arrows in FIGS. 5A and 5c show fluorescence indicating presence of labeled antibody in the cells.

FIGS. 6A-6D show internalization of 8D8 HA-lipid particles (prepared with Rhodamine-DPPE, 50 ul) into A549: Cells were stained with Concavalin A (1.5 μg/ml) and Hoechst reagents (1:10,000) for membrane and nuclei labeling respectively. 6A. cells incubated for 1 h at 37° C. with lipid particles only. 6B, 6C—incubated for 1 h at 37° C. with 8D8-coated lipid particles—specific internalization is detected. 6D. Incubation of 8D8 lipid particles at 4° C. (X525)—no entry is observed.

FIGS. 7A-7D shows internalization of 8D8 HA-lipid particles into NRK cells: Cells were stained with Concavalin A (1.5 ug/ml) and Hoechst reagents (1:10,000) for membrane and nuclei labeling respectively. 7A. NRK naive incubated for 1 h at 37° C. with lipid particles only. 7B. NRK naive incubated for 1 h at 37° C. with 8D8 lipid particles. 7C. NRK ENDO180 incubated for 1 h at 37° C. with 8D8 lipid particles. 7D. NRK-ENDO180 incubated for 1 h at 37° C. with HA-lipid particles only (X525). *NRK-ENDO180 cells contaminated with mycoplasma. FIG. 8 shows shift in fluorescence due to binding of lipid particle-antibody composition (8D8-NP) to NRK-ENDO180 cells when conjugation of antibody to lipid is via PEG spacer. IgG-Np refers to lipid particles conjugated to IgG antibodies.

FIG. 9 depicts reduced cell survival of ENDO180 expressing cells after specific delivery of doxorubicin (DOX) to NRK-ENDO180 cells via 8D8-NPs. Cell survival was measured using a XTT assay.

FIG. 10 shows binding of 8D8 AF 488-NPs to NRK-ENDO180.

FIGS. 11A and 11B show Cy3-siRNA delivery to NRK-ENDO180 expressing cells.

FIG. 12 shows Z-Stack images demonstrating uptake of Cy3-siRNA into NRK52-ENDO180 cells.

FIG. 13 shows Cy3-siRNA delivered via 8D8-NPs localized to the perinuclear foci (white arrows) where the RNAi machinery is also located.

FIG. 14 is a graph showing reduced cell survival of ENDO180+ cells using ENDO180-targeted lipid nanoparticles encapsulating MMC. XTT assay was performed 72 h post incubation. Each bar represent an average of 16 wells/treatment with the SD between the data points. The data presented is representative of three independent experiments.

FIGS. 15A and 15B show in vitro knock down of Rac1 mRNA (levels of residual mRNA shown) in a A549 cell line exposed to 8D8-NPs encapsulating siRNA to RAC (siRAC1).

FIGS. 16A-16D present graphs depicting biodistribution of siRNA to various body organs in mice treated with ENDO180 coated nanoparticles (NPs) encapsulating Cy5-Rac1_{—}28 in a murine cancer model. The amount of siRNA (atomoles) present per mg tissue sample is presented in animals treated with different compositions.

FIGS. 17A-17D present graphs depicting biodistribution of ENDO180 coated nanoparticles (NPs) encapsulating Rac1_{—}28 in the tumor and kidneys from a murine cancer model. The amount of siRNA (atomoles) present per mg tissue sample is presented in animals treated with different compositions.

### DETAILED DESCRIPTION OF THE INVENTION

### Definitions

For convenience certain terms employed in the specification, examples and claims are described herein.

It is to be noted that, as used herein, the singular forms “a”, “an” and “the” include plural forms unless the content clearly dictates otherwise.

Where aspects or embodiments are described in terms of Markush groups or other grouping of alternatives, those skilled in the art will recognize that the aspects or embodiments are also thereby described in terms of any individual member or subgroup of members of the group.

The terms “targeting agent” or “targeting moiety,” used interchangeably herein, refer to an agent that preferentially associates with or binds to a particular target which may include a specific cell type or tissue type, a protein including for example a receptor, an infecting agent or other target of interest. The targeting agent suitable for use in the disclosed compositions must have sufficient binding affinity for the target under physiological conditions to selectively recognize and bind to the appropriate cell type expressing the target by the desired delivery method (e.g. in vivo, in vitro, ex vivo). Examples of a targeting agent include, but are not limited to, an oligonucleotide including an aptamer, an antigen, an antibody or functional fragment thereof, a ligand, a receptor, one member of a specific binding pair, a polyamide including a peptide or peptidomimetic having affinity for a biological receptor, an oligosaccharide, a polysaccharide, a steroid or steroid derivative, a hormone, a hormone-mimic, e.g., morphine, or other compound having binding specificity for a target. In the methods disclosed herein, the targeting moiety promotes delivery of the delivery system to the target of interest, i.e., cells expressing the ENDO180 receptor.

The delivery system disclosed herein may utilize one or more different targeting agents. A plurality of targeting agents, each with their own binding target, on a particular delivery agent can be used to facilitate delivery to a broader spectrum of cell types (more than one cell type), or alternatively, to narrow the target cell type.

Antibodies and functional fragments or derivatives thereof which exhibit the desired binding activity (specifically bind the desired cell surface antigen) are useful targeting moieties. As used herein, an “antibody” or “functional fragment” of an antibody encompasses antibodies and derivatives thereof which exhibit the desired specific binding activity. This includes, without limitation, polyclonal and monoclonal antibodies, as well as preparations including hybrid or chimeric antibodies, such as humanized antibodies, altered antibodies, antibody fragments such as F(ab′)₂ fragments, F(ab) fragments, Fv fragments including ScFv, single domain antibodies, dimeric and trimeric antibody fragment constructs, minibodies, and functional fragments thereof which exhibit immunological binding properties of the parent antibody molecule and/or which bind a cell surface antigen, i.e. the ENDO180 receptor.

As disclosed herein a “lipid particle” may also be referred to as a “carrier moiety” and refers to without limitation, a lipid particle which may comprise non-lipid components. Disclosed herein are compositions comprising lipid particles. The composition disclosed herein includes a lipid particle, which has been modified by attachment of a targeting moiety.

As disclosed herein the lipid particle is also referred to herein as a lipid-based nanoparticle. Liposomes are closed lipid bilayer membranes containing an entrapped aqueous volume. Liposomes may be unilamellar vesicles (ULV) possessing a single membrane bilayer or multilameller vesicles (MLV), onion-like structures characterized by multiple membrane bilayers, each separated from the next by an aqueous layer. In one preferred embodiment, the lipid particles disclosed herein are unilamellar vesicles. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

The lipid-based nanoparticles disclosed herein may be produced from combinations of lipid materials well known and routinely utilized in the art to produce liposomes. Liposomes encompass relatively rigid types, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soybeans or other plant sources or those that are partially or wholly synthetic, or of variable lipid chain length and unsaturation are suitable for use, as disclosed herein. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in the compositions disclosed herein. All of these phospholipids are commercially available. Further, phosphatidylglycerols (PG) and phosphatic acid (PA) phosphatidylethanolamines (PE), are also suitable phospholipids for use in the compositions disclosed herein and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dioleoyl phosphatidylethanolamine (DOPE), dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in compositions. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol (Chol). Certain preferred lipids for producing the lipid-based nanoparticles disclosed herein include phosphatidylethanolamine (PE), dioleoyl phosphatidylethanolamine (DOPE) and phosphatidylcholine (PC) in further combination with cholesterol (CH). Other suitable lipids include the cationic lipids N-[1-(2,3-dioleyloxyl)propyl]-N,N,N-trimethylammonium chloride (DOTMA) and N-[1-(2,3-dioleoyloxy)propyl]-N,N,N-trimethylammonium chloride (DOTAP) and analogues thereof.

Non-cationic lipids include distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), a sterol (e.g., cholesterol) and a mixture thereof.

In some embodiments a combination of lipids and cholesterol for producing the liposomes disclosed herein comprise a PE:PC:Chol molar ratio of about 3:1:1 or about 4:2:1.

The lipid-based nanoparticles of the present invention may be obtained by any method known to the skilled artisan. For example, the lipid particle preparation disclosed herein can be produced by reverse phase evaporation (REV) method (see U.S. Pat. No. 4,235,871), infusion procedures, or detergent dilution. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). A method for forming ULVs is described in Cullis et al., PCT Publication No. 87/00238, Jan. 16, 1986, entitled “Extrusion Technique for Producing Unilamellar Vesicles”. Multilamellar liposomes (MLV) may be prepared by the lipid-film method, wherein the lipids are dissolved in a chloroform-methanol solution (3:1, vol/vol), evaporated to dryness under reduced pressure and hydrated by a swelling solution. Then, the solution is subjected to extensive agitation and incubation, for example 2 hours at 37° C. After incubation, unilamellar liposomes (ULV) are obtained by extrusion. The extrusion step modifies liposomes by reducing the size of the liposomes to a preferred and substantially uniform average diameter. Alternatively, liposomes of the desired size may be selected using techniques such as filtration or other size selection techniques. While the size-selected liposomes disclosed herein have an average diameter of less than about 200 nm, it is preferred that they are selected to have an average diameter of less than about 150 nm with an average diameter of about 90-150 nm being particularly preferred. When the lipid particle disclosed herein is a unilamellar liposome, it preferably is selected to have an average diameter of less than about 200 nm. The most preferred unilamellar lipid particles disclosed herein have an average diameter of less than about 150 nm.

The outer surface of the lipid-based nanoparticle may be modified to facilitate attachment of a targeting moiety. One example of such a modification is modification of the outer surface of the lipid-based nanoparticle with a natural or synthetic polymer, for example polyethylene glycol (PEG) or hyaluronic acid (HA). Other polymers include saccharides such as trehalose, sucrose, mannose or glucose. In one preferred embodiment, the lipid-based nanoparticle is coated with HA. Without wishing to be bound to theory, HA acts as both a long-circulating agent and a cryoprotectant. The polymer may be incorporated into the liposomal composition ab initio or may be combined with the prepared lipid-based nanoparticles.

The outer surface of the lipid-based nanoparticles may be further modified with an agent to enhance the uptake of the lipid-based nanoparticles into the tissue of interest and preclude or reduce uptake of the lipid-based nanoparticles into the cellular endothelial systems. The modification of the lipid-based nanoparticles with a hydrophilic polymer as the long-circulating agent prolongs the half-life of the lipid-based nanoparticle in the blood. Examples of hydrophilic polymers suitable for use include polyethylene glycol (PEG), polymethylethylene glycol, polyhydroxypropylene glycol, polypropylene glycol, polymethylpropylene glycol and polyhydroxypropylene oxide. Glycosaminoglycans, e.g., hyaluronic acid, may also be used as long-circulating agents.

The lipid-based nanoparticle is modified by attachment of the targeting moiety. In one embodiment, the targeting moiety is covalently conjugated to the cryoprotectant, e.g., HA. This can be accomplished using a crosslinking reagent (e.g. glutaraldehyde (GAD), bifunctional oxirane (OXR), ethylene glycol diglycidyl ether (EGDE), N-hydroxysuccinimide (NHS), and a water soluble carbodiimide, for example 1-ethyl-3-(3-dimethylaminopropyl)carbodiimide (EDC). As is known to the skilled artisan, any crosslinking chemistry can be used, including, but not limited to, thioether, thioester, malimide and thiol, amine-carboxyl, amine-amine, and others. Through crosslinking, linkage of the amine residues of the targeting moiety and lipid-based nanoparticles is established.

Modified lipid-based nanoparticles are prepared from empty micro- or nano-scale liposomes prepared by any method known to the skilled artisan from any liposome material known at the time. The lipid-based nanoparticle is preferably modified with a first layer surface modification by covalent binding. The first layer preferably comprises a polymer such as PEG or a glycosaminoglycan such as hyaluronic acid. To this, a second layer of surface modification may be added by covalent attachment to the first layer. The second layer includes a targeting agent or moiety as described herein, e.g., an antibody or functional fragment thereof. Further layers may add to the lipid-based nanoparticle additional agents (e.g. additional targeting moieties). Alternatively, the second layer may include a heterogeneous mix of targeting moieties. The lipid-based nanoparticle composition may be lyophilized after addition of the final layer. The therapeutic agent of interest may be encapsulated by the lipid-based nanoparticle by rehydration of the lipid-based nanoparticle with an aqueous solution containing the therapeutic agent or diagnostic agent. Therapeutic agents that are poorly soluble in aqueous solutions or agents that are hydrophobic may be added to the composition during preparation of the lipid-based nanoparticles. The lipid-based nanoparticle composition is optionally lyophilized and reconstituted at any time after the addition of the first layer.

In one embodiment, two agents of interest (e.g. therapeutic agents) may be delivered by the lipid particle. One agent can be hydrophobic and the other is hydrophilic. The hydrophobic agent may be added to the lipid particle during formation of the lipid particle. The hydrophobic agent associates with the lipid portion of the lipid particle. The hydrophilic agent is added in the aqueous solution rehydrating the lyophilized lipid particle. An exemplary embodiment of two-agent delivery is described below, wherein a condensed siRNA is encapsulated in a lipid-based nanoparticle and wherein a drug that is poorly soluble in aqueous solution is associated with the lipid portion of the lipid particle. As used herein, “poorly soluble in aqueous solution” refers to a composition that is less than about 10% soluble in water.

In addition to lipids, the lipid particle may further comprise additional agents comprising natural or synthetic polymers including a protein or non-protein polymer. Such lipid particles may be modified and enhanced similarly to the modifications described herein for the lipid-based nanoparticle carrier moieties. The lipid particle may further comprise a synthetic polymer such as poly(lactic acid) (PLA) and poly(lactic co-glycolic acid) (PLGA). In another embodiment, the composition further comprises a protein (e.g. a polypeptide) or the nucleic acid binding domain of a protein. In one embodiment, the binding moiety is the nucleic acid binding domain of a protein selected from the group of nucleic acid binding domains present in proteins selected from the group consisting of protamine, GCN4, Fos, Jun, TFIIS, FMRI, yeast protein HX, Vigillin, Merl, bacterial polynucleotide phosphorylase, ribosomal protein S3, and heat shock protein. In one embodiment, the binding moiety is the protein protamine or an RNA interference-inducing molecule-binding fragment of protamine.

An “inhibitor” is a compound, which is capable of reducing (partially or fully) the expression of a gene or the activity of the product of such gene to an extent sufficient to achieve a desired biological or physiological effect. The term “inhibitor” as used herein includes one or more of a nucleic acid inhibitor, including siRNA, shRNA, synthetic shRNA; miRNA, antisense RNA and DNA and ribozymes. An “inhibitory nucleic acid” includes an antisense compound, a chemically modified siRNA compound, an unmodified siRNA compound, a chemically modified shRNA compound, an unmodified shRNA compound, a chemically modified miRNA compound, and an unmodified miRNA compound.

A “siRNA inhibitor” is a compound capable of reducing the expression of a gene or the activity of the product of such gene to an extent sufficient to achieve a desired biological or physiological effect. The term “siRNA inhibitor” as used herein refers to one or more of a siRNA, shRNA, synthetic shRNA; miRNA. Inhibition may also be referred to as down-regulation or, for RNAi, silencing.

The term “inhibit” as used herein refers to reducing the expression of a gene or the activity of the product of such gene to an extent sufficient to achieve a desired biological or physiological effect. Inhibition may be complete or partial. As used herein, the term “ENDO180 gene” is defined as any homolog of the ENDO180 gene having preferably 90% homology, more preferably 95% homology, and even more preferably 98% homology to the amino acid encoding region of SEQ ID NO:1 or nucleic acid sequences which bind to the ENDO180 gene under conditions of highly stringent hybridization, which are well-known in the art (for example, see Ausubel et al., Current Protocols in Molecular Biology, John Wiley and Sons, Baltimore, Md. (1988), updated in 1995 and 1998).

As used herein, the term “ENDO180” or “ENDO180 polypeptide” or “ENDO180 receptor” is defined as any homolog of the ENDO180 polypeptide having preferably at least 90% homology, more preferably at least 95% homology, and even more preferably at least 98% homology or 100% identity to SEQ ID NO:2, as either full-length or a fragments or a domain thereof, as a mutant or the polypeptide encoded by a spliced variant nucleic acid sequence, as a chimera with other polypeptides, provided that any of the above has the same or substantially the same biological function as the ENDO180 receptor. ENDO180 polypeptide, or an ENDO180 polypeptide homolog, may be present in different forms, including but not limited to soluble protein, membrane-bound (either in purified membrane preparations or on a cell surface), bead-bound, or any other form presenting ENDO180 protein or fragments and polypeptides derived thereof. The term “inhibit” as used herein refers to reducing the expression of a gene or the activity of the product of such gene to an extent sufficient to achieve a desired biological or physiological effect. Inhibition is either complete or partial.

The terms “mRNA polynucleotide sequence”, “mRNA sequence” and “mRNA” are used interchangeably.

As used herein, the terms “polynucleotide” and “nucleic acid” may be used interchangeably and refer to nucleotide sequences comprising deoxyribonucleic acid (DNA), or ribonucleic acid (RNA). The terms are to be understood to include, as equivalents, analogs of either RNA or DNA made from nucleotide analogs. Throughout this application, mRNA sequences are set forth as representing the corresponding genes.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The nucleic acid molecules disclosed herein encompass molecules comprising deoxyribonucleotides, ribonucleotides, modified deoxyribonucleotides, modified ribonucleotides and combinations thereof.

As used herein, the term “nucleic acid molecule” or “nucleic acid” are used interchangeably and refer to an oligonucleotide, nucleotide or polynucleotide. Variations of “nucleic acid molecule” are described in more detail herein. A nucleic acid molecule encompasses both single stranded (i.e. antisense) and double stranded molecules (i.e. dsRNA, siRNA), both modified nucleic acid molecules and unmodified nucleic acid molecules as described herein. A nucleic acid molecule may include deoxyribonucleotides, ribonucleotides, modified nucleotides or nucleotide analogs in any combination.

“Substantially complementary” refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly “substantially identical” refers to identity of greater than about 84%, to another sequence.

The “linker” as disclosed herein is a nucleotide or non-nucleotide moiety which links, for example, the antibody to the therapeutic molecule, or the antibody to the lipid, or the antibody to the GAG, or the GAG to the lipid. In some embodiments the linker is a cleavable moiety. Preferred cleavable groups include a disulfide bond, amide bond, thioamide, bond, ester bond, thioester bond, vicinal diol bond, or hemiacetal. Other cleavable bonds include enzymatically-cleavable bonds, such as peptide bonds (cleaved by peptidases), phosphate bonds (cleaved by phosphatases), nucleic acid bonds (cleaved by endonucleases), and sugar bonds (cleaved by glycosidases).

In some embodiments the linker is a non-nucleotide linker including a peptide linker. The choice of peptide sequence is critical to the success of the conjugate. In some embodiments the linker is stable to serum proteases, yet is cleaved by the lysosomal enzymes in the target cell. In a non-limiting example the linker is a peptide selected from a linker set forth in U.S. Pat. No. 5,574,142, protamine, a fragment of protamine, (Arg)9, biotin-avidin, biotin-streptavidin and antennapedia peptide. For example, a peptide linker is used to link the antibody to a nucleic acid based therapeutic agent. Other non-nucleotide linkers include alkyl or aryl chains of about 5 to about 100 atoms.

In some embodiments the linker is a nucleotide linker. In certain embodiments a nucleic acid linker has a length ranging from 2-100, preferably 2-50 or 2-30 nucleotides.

### Oligonucleotide Chemical Modifications

In some embodiments the therapeutic and/or diagnostic agent comprises an oligonucleotide molecule. In some embodiments the oligonucleotide is single stranded or double stranded. In some embodiments the oligonucleotide is an antisense or RNAi agent.

“Nucleotide” is meant to encompass deoxyribonucleotides and ribonucleotides, which may be natural or synthetic, and/or modified or unmodified. Modifications include changes to the sugar moiety, the base moiety and/or the linkages between ribonucleotides in the oligoribonucleotide. As used herein, the term “ribonucleotide” encompasses natural and synthetic, unmodified and modified ribonucleotides. Modifications include changes to the sugar moiety, to the base moiety and/or to the linkages between ribonucleotides in the oligonucleotide.

The nucleotides useful in preparing a therapeutic agent (i.e. a nucleic acid molecule) include naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine. In some embodiments one or more nucleotides in an oligomer is substituted with inosine.

According to some embodiments provided herein are inhibitory oligonucleotide compounds comprising unmodified and modified nucleotides and/or unconventional moieties. In certain embodiments the therapeutic agent is an oligonucleotide/nucleic acid molecule. In various preferred embodiments the therapeutic agent is a double stranded oligonucleotide and preferably siRNA. In some embodiments a chemically modified siRNA molecule is preferred.

The selection and synthesis of siRNA corresponding to known genes has been widely reported; (see for example Ui-Tei et al., 2006. J Biomed Biotechnol.; 2006:65052; Chalk et al., 2004. BBRC. 319(1): 264-74; Sioud & Leirdal, 2004. Met. Mol Biol.; 252:457-69; Levenkova et al., 2004, Bioinform. 20(3):430-2; Ui-Tei et al., 2004. NAR 32(3):936-48).

For examples of the use of, and production of, modified siRNA see for example Braasch et al., 2003. Biochem., 42(26):7967-75; Chiu et al., 2003, RNA, 9(9):1034-48; PCT publications WO 2004/015107 (atugen AG) and WO 02/44321 (Tuschl et al). U.S. Pat. Nos. 5,898,031 and 6,107,094 teach chemically modified oligomers. U.S. Pat. No. 7,452,987 relates to oligomeric compounds having alternating unmodified and 2′ sugar modified ribonucleotides. US patent publication No. 2005/0042647 describes dsRNA compounds having chemically modified internucleoside linkages.

Amarzguioui et al., (2003, NAR, 31(2):589-595) showed that siRNA activity depended on the positioning of the 2′-O-methyl modifications. Holen et al (2003, NAR, 31(9):2401-2407) report that an siRNA having small numbers of 2′-O-methyl modified nucleosides showed good activity compared to wild type but that the activity decreased as the numbers of 2′-O-methyl modified nucleosides was increased. Chiu and Rana (2003, RNA, 9:1034-1048) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., 2003, NAR, 31(11), 2705-2716).

PCT Patent Application Nos. PCT/IL2008/000248 and PCT/IL2008/001197, are hereby incorporated by reference in their entirety disclose motifs useful in the preparation of chemically modified siRNA compounds. PCT Patent Publication No. WO 2008/020435 discloses inhibitors, including some siRNA compounds to the target genes set forth herein.

The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid), PNA (peptide nucleic acid), arabinoside, phosphonocarboxylate or phosphinocarboxylate nucleotide (PACE nucleotide), mirror nucleotide, or nucleotides with a 6 carbon sugar.

All analogs of, or modifications to, a nucleotide/oligonucleotide may be employed with the compositions disclosed herein, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

A sugar modification includes a modification on the 2′ moiety of the sugar residue and encompasses amino, fluoro, alkoxy e.g. methoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1.

In one embodiment the siRNA compound comprises at least one ribonucleotide comprising a 2′ modification on the sugar moiety (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities but may also contain thiophosphate-D-ribose entities, triester, thioate, 2′-5′ bridged backbone (also may be referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide. In addition, analogs of polynucleotides may be prepared wherein the structure of one or more nucleotide is fundamentally altered and better suited as therapeutic or experimental reagents. An example of a nucleotide analog is a peptide nucleic acid (PNA) wherein the deoxyribose (or ribose) phosphate backbone in DNA (or RNA is replaced with a polyamide backbone which is similar to that found in peptides. PNA analogs have been shown to be resistant to enzymatic degradation and to have extended stability in vivo and in vitro. Other modifications that can be made to oligonucleotides include polymer backbones, cyclic backbones, acyclic backbones, thiophosphate-D-ribose backbones, triester backbones, thioate backbones, 2′-5′ bridged backbone, artificial nucleic acids, morpholino nucleic acids, glycol nucleic acid (GNA), threose nucleic acid (TNA), arabinoside, and mirror nucleoside (for example, beta-L-deoxyribonucleoside instead of beta-D-deoxyribonucleoside). Examples of siRNA compounds comprising LNA nucleotides are disclosed in Elmen et al., (NAR 2005, 33(1):439-447).

Additional modifications to the oligonucleotides include the presence of nucleotide and or non-nucleotide moieties at one or more of the termini.

The compounds of the present nucleic acid molecules disclosed herein may be synthesized using one or more inverted nucleotides, for example inverted thymidine or inverted adenine (see, for example, Takei, et al., 2002, JBC 277(26):23800-06).

What is sometimes referred to herein as an “abasic nucleotide” or “abasic nucleotide analog” is more properly referred to as a pseudo-nucleotide or an unconventional moiety. A nucleotide is a monomeric unit of nucleic acid, consisting of a ribose or deoxyribose sugar, a phosphate, and a base (adenine, guanine, thymine, or cytosine in DNA; adenine, guanine, uracil, or cytosine in RNA). A modified nucleotide comprises a modification in one or more of the sugar, phosphate and or base. The abasic pseudo-nucleotide lacks a base, and thus is not strictly a nucleotide.

Other modifications include terminal modifications selected from a nucleotide, a modified nucleotide, a lipid, a peptide, a sugar and inverted abasic moiety.

In some embodiments the siRNA therapeutic agent comprises a capping moiety. The term “capping moiety” as used herein includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′O-Me nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Certain preferred capping moieties are abasic ribose or abasic deoxyribose moieties; inverted abasic ribose or abasic deoxyribose moieties; C6-amino-Pi; a mirror nucleotide including L-DNA and L-RNA.

In some embodiments the therapeutic siRNA comprises a moiety other than a nucleotide. The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; bridged nucleic acids including LNA and ethylene bridged nucleic acids.

A “mirror” nucleotide is a nucleotide with reversed chirality to the naturally occurring or commonly employed nucleotide, i.e., a mirror image (L-nucleotide) of the naturally occurring (D-nucleotide), also referred to as L-RNA in the case of a mirror ribonucleotide, and “spiegelmer”. The nucleotide can be a ribonucleotide or a deoxyribonucleotide and my further comprise at least one sugar, base and or backbone modification. See U.S. Pat. No. 6,586,238. Also, U.S. Pat. No. 6,602,858 discloses nucleic acid catalysts comprising at least one L-nucleotide substitution. Mirror nucleotide includes for example L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU).

Modified deoxyribonucleotide includes, for example 5′OMe DNA (5-methyl-deoxyriboguanosine-3′-phosphate) which may be useful as a nucleotide in the 5′ terminal position (position number 1); PACE (deoxyriboadenine 3′ phosphonoacetate, deoxyribocytidine 3′ phosphonoacetate, deoxyriboguanosine 3′ phosphonoacetate, deoxyribothymidine 3′ phosphonoacetate.

Bridged nucleic acids include LNA (2′-O,4′-C-methylene bridged Nucleic Acid adenosine 3′ monophosphate, 2′-O,4′-C-methylene bridged Nucleic Acid 5-methyl-cytidine 3′ monophosphate, 2′-O,4′-C-methylene bridged Nucleic Acid guanosine 3′ monophosphate, 5-methyl-uridine (or thymidine) 3′ monophosphate); and ENA (2′-O,4′-C-ethylene bridged Nucleic Acid adenosine 3′ monophosphate, 2′-O,4′-C-ethylene bridged Nucleic Acid 5-methyl-cytidine 3′ monophosphate, 2′-O,4′-C-ethylene bridged Nucleic Acid guanosine 3′ monophosphate, 5-methyl-uridine (or thymidine) 3′ monophosphate).

According to one aspect, provided herein inhibitory oligonucleotide compounds comprising unmodified and modified nucleotides. The compound comprises at least one modified nucleotide selected from the group consisting of a sugar modification, a base modification and an internucleotide linkage modification and may contain DNA, and modified nucleotides such as LNA (locked nucleic acid) including ENA (ethylene-bridged nucleic acid; PNA (peptide nucleic acid); arabinoside; PACE (phosphonoacetate and derivatives thereof), mirror nucleotide, or nucleotides with a six-carbon sugar. In some embodiments the present provided herein are methods and compositions for inhibiting expression of a target gene in vivo. In general, the method includes administering a delivery-therapeutic agent conjugate. In particular embodiments the conjugate comprises small interfering RNAs (i.e. siRNAs), that target an mRNA transcribed from the target gene in an amount sufficient to down-regulate expression (reduce mRNA levels, reduce protein levels) of a target gene, for example by an RNA interference (RNAi) mechanism. In particular, the subject method can be used to inhibit expression of the target gene for treatment of a disease. The nucleic acid molecules to the target gene are useful as therapeutic agents to treat various pathologies. In one embodiment the nucleic acid molecule down-regulaties a target polypeptide, whereby the down-regulation of the target polypeptide includes down-regulation of target polypeptide function (which may be examined, for example, by an enzymatic assay or a binding assay with a known interactor of the native gene/polypeptide), down-regulation of target protein (which may be examined, for example, by Western blotting, ELISA or immuno-precipitation) and down-regulation of target polypeptide mRNA expression (which may be examined by Northern blotting, quantitative RT-PCR, in-situ hybridisation or microarray hybridisation, RACE).

The synthesis of the nucleic acid molecules described herein, is within the skills of the one of the art. Such synthesis is, among others, described in Beaucage S L and Iyer R P, 1992 Tetrahedron; 48: 2223-2311, Beaucage S. and Iyer R P, 1993 Tetrahedron; 49: 6123-6194 and Caruthers M H et. al., 1987 Methods Enzymol.; 154: 287-313, the synthesis of thioates is, among others, described in Eckstein F., 1985 Annu. Rev. Biochem.; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

siRNA for any one of the target genes is synthesized using methods known in the art as described above, based on the known sequence of the target mRNA and is stabilized to serum and/or cellular nucleases by various modifications as described herein.

### Target Genes

The delivery system disclosed herein is useful for delivery of a therapeutic agent and/or a diagnostic agent to a cell expressing ENDO180. In some embodiments the therapeutic agent comprises an anti-cell proliferative agent.

In some embodiments the therapeutic agent comprises a nucleic acid compound which inhibits a target gene or expression of a target gene, the target gene associated with a disease or disorder selected from the group consisting of a proliferative disease, a metastatic disease, and fibrosis.

Target genes include anti-apoptotic genes, genes associated with basic cell division machinery, genes associated with cell cycle regulation/cell proliferation, genes associated with rate-limiting metabolism (nucleotide/nucleic acid synthesis, protein synthesis, energy metabolism), genes associated with protein trafficking (e.g., secretion); pro-inflammatory genes, cytokines, chemokines, NFkB, growth factors/receptors (TGFβ1 and 2, CTGF, IGF1, PDGF1, PDGF2, VEGF, EGFR, HER2, etc), genes associated with fibrosis (HSP47, TGFβ1, IL-10).

Sense and antisense sequences useful in the synthesis of siRNA are selected according to proprietary and publicly available methods and algorithms.

The chemical modifications provided above are useful in synthesizing nucleotide therapeutics that exhibit inter alia, serum stability, activity, reduced immune response, reduced off target effect.

### Antibodies

The term “antibody” refers to IgG, IgM, IgD, IgA, and IgE antibody, inter alia. The definition includes polyclonal antibodies or monoclonal antibodies. This term refers to whole antibodies or fragments of antibodies comprising an antigen-binding domain, e.g. antibodies without the Fc portion, single chain antibodies, miniantibodies, fragments consisting of essentially only the variable, antigen-binding domain of the antibody, etc. The term “antibody” may also refer to antibodies against polynucleotide sequences obtained by cDNA vaccination. The term also encompasses antibody fragments which retain the ability to selectively bind with their antigen or receptor and are exemplified as follows, inter alia:

(1) Fab, the fragment which contains a monovalent antigen-binding fragment of an antibody molecule which can be produced by digestion of whole antibody with the enzyme papain to yield a light chain and a portion of the heavy chain;

(2) (Fab′)2, the fragment of the antibody that can be obtained by treating whole antibody with the enzyme pepsin without subsequent reduction; F(ab′2) is a dimer of two Fab fragments held together by two disulfide bonds;

(3) Fv, defined as a genetically engineered fragment containing the variable region of the light chain and the variable region of the heavy chain expressed as two chains; and

(4) Single chain antibody (SCA), defined as a genetically engineered molecule containing the variable region of the light chain and the variable region of the heavy chain linked by a suitable polypeptide linker as a genetically fused single chain molecule, including a scFv.

CDR grafting may be performed to alter certain properties of the antibody molecule including affinity or specificity. A non-limiting example of CDR grafting is disclosed in U.S. Pat. No. 5,225,539.

Single-domain antibodies are isolated from the unique heavy-chain antibodies of immunized Camelidae, including camels and llamas. The small antibodies are very robust and bind the antigen with high affinity in a monomeric state. U.S. Pat. No. 6,838,254 describes the production of antibodies or fragments thereof derived from heavy chain immunoglobulins of Camelidae.

A monoclonal antibody (mAb) is a substantially homogeneous population of antibodies to a specific antigen. Monoclonal antibodies (mAbs) are obtained by methods known to those skilled in the art. See, for example Kohler et al (1975); U.S. Pat. No. 4,376,110; Ausubel et al (1987-1999); Harlow et al (1988); and Colligan et al (1993), the contents of which are incorporated entirely herein by reference. The mAbs disclosed herein may be of any immunoglobulin class including IgG, IgM, IgE, IgA, and any subclass thereof. A hybridoma producing a mAb may be cultivated in vitro or in vivo. High titers of mAbs are obtained in vivo for example wherein cells from the individual hybridomas are injected intraperitoneally into pristine-primed Balb/c mice to produce ascites fluid containing high concentrations of the desired mAbs. mAbs of isotype IgM or IgG may be purified from such ascites fluid, or from culture supernatants, using column chromatography methods well known to those of skill in the art.

By “specific binding affinity” is meant that the antibody binds to an ENDO180 polypeptide or fragment thereof with greater affinity than it binds to another polypeptide under similar conditions.

The term “epitope” is meant to refer to that portion of a molecule capable of being bound by an antibody which can also be recognized by that antibody. An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

Epitopes or antigenic determinants usually consist of chemically active surface groupings of molecules such as amino acids or sugar side chains and have specific three-dimensional structural characteristics as well as specific charge characteristics.

In one embodiment, the antibody is a monoclonal antibody. In one embodiment, the monoclonal antibody is an IgG, IgM, IgD, IgA, or IgE monoclonal antibody. IgG subclasses are also well known to those in the art and include but are not limited to human IgG1, IgG2, IgG3 and IgG4. In one embodiment the monoclonal antibody is an IgG monoclonal antibody. In one embodiment, the monoclonal antibody is a human, humanized, or chimeric, antibody. In one embodiment, the portion of the antibody is a Fab fragment of the antibody. In one embodiment, the portion of the antibody comprises the variable domain of the antibody. In one embodiment, the portion of the antibody comprises a CDR portion of the antibody. In other embodiments the antibody is a scFv molecule. The antibodies may be produced recombinantly (see generally Marshak et al., 1996 “Strategies for Protein Purification and Characterization. A laboratory course manual.” Plainview, N.Y.: Cold Spring Harbor Laboratory Press, 1996) and analogs may be produced by post-translational processing. Differences in glycosylation can provide polypeptide analogs.

The antibody may be a human or nonhuman antibody. A nonhuman antibody may be humanized by recombinant methods to reduce its immunogenicity in man. Methods for humanizing antibodies are known to those skilled in the art.

This application provides humanized forms of the above antibodies. As used herein, “humanized” describes antibodies wherein some, most or all of the amino acids outside the CDR regions are replaced with corresponding amino acids derived from human immunoglobulin molecules, e.g. the human framework regions replace the non-human regions. In one embodiment of the humanized forms of the antibodies, some, most or all of the amino acids outside the CDR regions have been replaced with amino acids from human immunoglobulin molecules but where some, most or all amino acids within one or more CDR regions remain unchanged. Small additions, deletions, insertions, substitutions or modifications of amino acids are permissible as long as they would not abrogate the ability of the antibody to bind the antigen, ENDO180.

A “humanized” antibody would retain a similar antigenic specificity as the original antibody, i.e. the ability to bind ENDO180, specifically human ENDO180 receptor and would similarly be internalized by the receptor.

One skilled in the art would know how to produce the humanized antibodies of the subject invention. Various publications, several of which are hereby incorporated by reference into this application, describe how to make humanized antibodies.

For example, the methods described in U.S. Pat. Nos. 4,816,567 and 6,331,415 comprise the production of chimeric antibodies having a variable region of one antibody and a constant region of another antibody.

U.S. Pat. Nos. 5,225,539; 6,548,640 and 6,982,321 describes the use of recombinant DNA technology to produce a humanized antibody wherein the CDRs of one immunoglobulin are replaced with the CDRs from an immunoglobulin with a different specificity such that the humanized antibody would recognize the target antigen but would not illicit an immune response. Specifically, site directed mutagenesis is used to introduce the CDRs onto the framework region.

Other approaches for humanizing an antibody are described in WO 90/07861 and corresponding patents including U.S. Pat. Nos. 5,585,089; 5,693,761; 6,180,370 and 7,022,500. These patents describe a method to increase the affinity of an antibody for the desired antigen by combining the CDRs of a mouse monoclonal antibody with human immunoglobulin framework and constant regions. Human framework regions can be chosen to maximize homology with the mouse sequence. Computer modeling can be used to identify amino acids in the framework region which are likely to interact with the CDRs or the specific antigen and then mouse amino acids can be used at these positions to create the humanized antibody.

The above methods are merely illustrative of some of the methods that one skilled in the art could employ to make humanized antibodies.

The monoclonal antibody E3-8D8 represents a suitable anti-ENDO180 antibody for use in the compositions and methods disclosed herein. The hybridoma cell E3-8D8 was deposited with the Belgian Co-ordinated Collections of Micro-Organisms (BCCM), under the terms of the Budapest Treaty and given Accession Number LMBP 7203CB.

### Epitope Mapping

Epitope mapping studies identify the residues that are important for antibody binding. Various methods are known in the art for epitope mapping and are readily performed by one skilled in the art. Certain methods are described in Epitope Mapping: A Practical Approach (0. M. R. Westwood, F. C. Hay; Oxford University Press, 2000), incorporated herein by reference.

One example of an epitope mapping techniques is Synthetic Labeled Peptides Epitope Mapping whereby a set of overlapping synthetic peptides is synthesized, each corresponding to a small segment of the linear sequence of the protein antigen, i.e. extracellular domain of ENDO180, and arrayed on a solid phase. The panel of peptides is then probed with the test antibody, and bound antibody is detected using an enzyme-labeled secondary antibody.

Other techniques include fragmentation or cleavage and gel separation of the protein antigen, transfer to a membrane, probing by test antibody and bound antibody is detected using an enzyme-labeled secondary antibody.

### Antibody Drug Development

In general monoclonal antibodies need to be designed to preserve binding properties (selectivity, internalization etc) and to reduce an immune response in the recipient. Specifically, the monoclonal antibody secreted from hybridoma 3E-8D8 may be optimized for human therapeutics by one of several methods known to those with skill in the art. In one method the variable heavy chain (V_{H}) and variable light chain (V_{L}) of the monoclonal antibody are sequenced. Once the amino acid sequence is known, the complementarity determining regions (CDR), heavy chain and light chain CDR3 are identified and degenerate oligonucleotides are used to clone synthetic CDR3 into a vector to produce a recombinant vector or construct. The construct may be for example a Fab fragment, a F(ab′)2 fragment, a Fv fragment, a single chain fragment or a full IgG molecule. The construct(s) is expressed and a polypeptide is isolated. In some embodiments the monoclonal antibody may be further optimized by mutagenesis optimized by site directed mutagenesis to generate a CDR3 domain having substantial identity to the original CDR3.

### Therapeutic Agents

The therapeutic agents or active agents useful in preparing and using the compositions disclosed herein include nucleotide and non-nucleotide agents, including oligonucleotides such as antisense (AS), miRNA and unmodified and chemically modified siRNA compounds. A preferred therapeutic agent is a siRNA compound.

In some embodiments the siRNA targets and reduces expression of a target gene by RNA interference.

The compositions and methods disclosed herein are useful for the treatment or diagnosis of diseases that arise from or otherwise involve aberrant cell proliferation. A therapeutic agent as the term is used herein, is an agent, which when delivered to a target cell, effects the target cell in such a way as to contribute to treatment of subject suffering from a disease, i.e. alleviation or amelioration of symptoms of a disease in the recipient subject. As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms; or relieving the disease, i.e., causing regression of the disease or its clinical symptoms. A therapeutic agent may also be an agent useful for diagnosis of disease or disease progression or of effects of treatment of the disease.

In one embodiment, the compositions are administered to a subject exhibiting aberrant cell proliferation in one or more organs.

Useful therapeutic agents include nucleic acids, small molecules, polypeptides, antibodies or functional fragments thereof. These core components as therapeutic agents may be further by modified to enhance function or storage, (e.g. enhance cellular uptake, increase specificity for the target, increase half-life, facilitate generation or storage). Nucleic acid therapeutic agents include DNA and RNA molecules, both single- and double-stranded. More than one therapeutic agent may be delivered by the compositions disclosed herein.

Therapeutic agents delivered by the methods disclosed herein include small molecules and peptides to block intracellular signaling cascades, enzymes (kinases), proteosome, lipid metabolism, cell cycle, membrane trafficking. Therapeutic agents delivered by the compositions and methods disclosed herein include chemotherapy agents.

The therapeutic agents may be associated with the lipid particle by any method known to the skilled artisan and includes, without limitation, encapsulation in the interior, association with the lipid portion of the molecule or association with the exterior of the lipid particle. Small molecule drugs soluble in aqueous solution may be encapsulated in the interior of the lipid particle. Small molecule drugs that are poorly soluble in aqueous solution may associate with the lipid portion of the lipid particle. Nucleic acid based therapeutic agents may associate with the exterior of the lipid particle. Such nucleic acids may be condensed with cationic polymers, e.g., PEI, or cationic peptides, e.g., protamines, and encapsulated in the interior of the lipid particle. Therapeutic peptides may be encapsulated in the interior of the lipid particle. Therapeutic peptides may be covalently attached to the exterior of the lipid particle.

In embodiments where the therapeutic agent is a nucleic acid, a lipid particle is particularly suitable for nucleic acid transport.

In one embodiment, the therapeutic agent is a nucleic acid, such as an RNA or DNA molecule (e.g. a double stranded RNA or single stranded DNA oligonucleotide). Useful DNA molecules are antisense as well as sense (e.g. coding and/or regulatory) DNA. Antisense DNA molecules include short oligonucleotides. Useful RNA molecules include RNA interference molecules, of which there are several known types. The field of RNA interference molecules has greatly expanded in recent years. Examples of useful RNA interference molecules are dsRNA including siRNA, siNA, shRNA, and miRNA (e.g., short temporal RNAs and small modulatory RNAs (Kim. 2005. Mol. Cells. 19:1-15)). As used herein, “double stranded RNA” or “dsRNA” refers to RNA molecules that are comprised of two strands. The therapeutic oligonucleotides disclosed herein are synthesized by any method known in the art for ribonucleic or deoxyribonucleic nucleotides. For example, a commercial polynucleotide synthesizer (e.g. Applied Biosystems 380B DNA synthesizer) can be used. When fragments are used, two or more such sequences can be synthesized and linked together for use in the compositions disclosed herein.

In some embodiments the therapeutic agent is selected from alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analog topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogs); podophyllotoxin; podophyllinic acid; teniposide; cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 1994. 33: 183-186); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN8); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle composition of paclitaxel (ABRAXANE™), and doxetaxel (TAXOTERE®); chloranbucil; 6-thioguanine; mercaptopurine; methotrexate; a platinum analog such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); a retinoid such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN®) combined with 5-FU and leucovovin.

Also included in this definition are anti-hormonal agents that act to regulate, reduce, block, or inhibit the effects of hormones that can promote the growth of cancer, and are often administered as systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); siRNA, ribozyme and antisense oligonucleotides, particularly those that inhibit expression of genes in signaling pathways implicated in aberrant cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl)benzenesulfonamide; and pharmaceutically acceptable salts, acids or derivatives of any of the above.

As used herein, the term “polypeptide” refers to, in addition to a polypeptide, a peptide and a full protein and includes isolated and recombinant polypeptides. As used herein, “biological function” refers to the biological property of the molecule and in this context means an in vivo effector or antigenic function or activity that is directly or indirectly performed by a naturally occurring polypeptide or nucleic acid molecule. Biological functions include but are not limited to receptor binding, any enzymatic activity or enzyme modulatory activity, any carrier binding activity, any hormonal activity, any activity in internalizing molecules or translocation from one compartment to another, any activity in promoting or inhibiting adhesion of cells to extracellular matrix or cell surface molecules, or any structural role, as well as having the nucleic acid sequence encode functional protein and be expressible. The antigenic functions essentially mean the possession of an epitope or an antigenic site that is capable of cross-reacting with antibodies raised against a naturally occurring protein. Biologically active analogs share an effector function of the native polypeptide that may, but need not, in addition possess an antigenic function.

Measurement of the level of the ENDO180 polypeptide may be determined by a method selected from the group consisting of immunohistochemistry, western blotting, ELISA, antibody microarray hybridization and targeted molecular imaging. Such methods are well-known in the art, for example immunohistochemistry, western blotting, antibody microarray hybridization, and targeted molecular imaging.

Measurement of the level of ENDO180 polynucleotide may be determined by a method selected for example from: RT-PCR analysis, in-situ hybridization, polynucleotide microarray and Northern blotting. Such methods are well known in the art.

### Antisense Molecules

In some embodiments the therapeutic agent is an antisense oligonucleotide. By the term “antisense” (AS) or “antisense fragment” is meant a polynucleotide fragment (comprising either deoxyribonucleotides, ribonucleotides or a mixture of both) having inhibitory antisense activity, said activity causing a decrease in the expression of the endogenous genomic copy of the corresponding gene. An AS polynucleotide is a polynucleotide which comprises consecutive nucleotides having a sequence of sufficient length and homology to a sequence present within the sequence of the target gene to permit hybridization of the AS to the gene. Many reviews have covered the main aspects of antisense (AS) technology and its therapeutic potential (Aboul-Fadl T., Curr Med Chem. 2005, 12(19):2193-214; Crooke S T, Curr MoI Med. 2004, 4(5):465-87; Crooke S T, Ann Rev Med. 2004, 55:61-95; Vacek M et al, Cell Mol Life Sci. 2003, 60(5):825-33; Cho-Chung Y S, Arch Pharm Res. 2003, 26(3): 183-91. There are further reviews on the chemical (Crooke et al., Hematol Pathol. 1995, 9(2):59-72), cellular (Wagner, Nature. 1994, 372(6504):333-5) and therapeutic (Scanlon, et al, FASEB J. 1995, 9(13): 1288-96) aspects of AS technology. Antisense intervention in the expression of specific genes can be achieved by the use of modified AS oligonucleotide sequences (for recent reports see Lefebvre-d′Hellencourt et al, 1995; Agrawal, 1996; LevLehman et al, 1997).

AS oligonucleotide sequences may be short sequences of DNA, typically 15-30 mer but may be as small as 7-mer (Wagner et al, Nat. Biotech. 1996, 14(7):840-4), designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, Semin Oncol. 1996, 23(1):78-87). In that case, RNaseH will cleave the RNA component of the duplex and can potentially release the AS to further hybridize with additional molecules of the target RNA. An additional mode of action results from the interaction of AS with genomic DNA to form a triple helix, which can be transcriptionally inactive.

The sequence target segment for the antisense oligonucleotide is selected such that the sequence exhibits suitable energy related characteristics important for oligonucleotide duplex formation with their complementary templates, and shows a low potential for self-dimerization or self-complementation (Anazodo et al, 1996, BBRC. 229:305-309). For example, the computer program OLIGO (Primer Analysis Software, Version 3.4), can be used to determine antisense sequence melting temperature, free energy properties, and to estimate potential self-dimer formation and self-complimentary properties. The program allows the determination of a qualitative estimation of these two parameters (potential self-dimer formation and self-complimentary) and provides an indication of “no potential” or “some potential” or “essentially complete potential”. Using this program target segments are generally selected that have estimates of no potential in these parameters. However, segments can be used that have “some potential” in one of the categories. A balance of the parameters is used in the selection as is known in the art. Further, the oligonucleotides are also selected as needed so that analog substitution does not substantially affect function.

Phosphorothioate antisense oligonucleotides do not normally show significant toxicity at concentrations that are effective and exhibit sufficient pharmacodynamic half-lives in animals (Agrawal, et al., PNAS USA. 1997, 94(6):2620-5) and are nuclease resistant. Antisense oligonucleotide inhibition of basic fibroblast growth factor (bFGF), having mitogenic and angiogenic properties, suppressed 80% of growth in glioma cells (Morrison, J Biol Chem. 1991 266(2):728-34) in a saturable and specific manner. Being hydrophobic, antisense oligonucleotides interact well with phospholipid membranes (Akhter et al., NAR. 1991, 19:5551-5559). Following their interaction with the cellular plasma membrane, they are actively (or passively) transported into living cells (Loke et al., PNAS 1989, 86(10):3474-8), in a saturable mechanism predicted to involve specific receptors (Yakubov et al., PNAS, 1989 86(17):6454-58).

siRNA and RNA Interference

RNA interference (RNAi) is a phenomenon involving double-stranded (ds) RNA-dependent gene-specific posttranscriptional silencing. Initial attempts to study this phenomenon and to manipulate mammalian cells experimentally were frustrated by an active, non-specific antiviral defense mechanism which was activated in response to long dsRNA molecules (Gil et al., Apoptosis, 2000. 5:107-114). Later, it was discovered that synthetic duplexes of 21 nucleotide RNAs could mediate gene specific RNAi in mammalian cells, without stimulating the generic antiviral defense mechanisms Elbashir et al. Nature 2001, 411:494-498 and Caplen et al. PNAS 2001, 98:9742-9747). As a result, small interfering RNAs (siRNAs), which are short double-stranded RNAs, have been widely used to inhibit gene expression and understand gene function.

RNA interference (RNAi) is mediated by small interfering RNAs (siRNAs) (Fire et al, Nature 1998, 391:806) or microRNAs (miRNAs) (Ambros V. Nature 2004, 431:350-355); and Bartel D P. Cell. 2004 116(2):281-97). The corresponding process is commonly referred to as specific post-transcriptional gene silencing when observed in plants and as quelling when observed in fungi.

A siRNA is a double-stranded RNA which down-regulates or silences (i.e. fully or partially inhibits) the expression of an endogenous or exogenous gene/mRNA. RNA interference is based on the ability of certain dsRNA species to enter a specific protein complex, where they are then targeted to complementary cellular RNA (i.e. mRNA), which they specifically degrade or cleave. Thus, the RNA interference response features an endonuclease complex containing siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir, et al., Genes Dev., 2001, 15:188). In more detail, longer dsRNAs are digested into short (17-29 bp) dsRNA fragments (also referred to as short inhibitory RNAs or “siRNAs”) by type III RNAses (DICER, DROSHA, etc., (see Bernstein et al., Nature, 2001, 409:363-6 and Lee et al., Nature, 2003, 425:415-9). The RISC protein complex recognizes these fragments and complementary mRNA. The whole process is culminated by endonuclease cleavage of target mRNA (McManus and Sharp, Nature Rev Genet, 2002, 3:737-47; Paddison and Hannon, Curr Opin Mol Ther. 2003, 5(3): 217-24). (For additional information on these terms and proposed mechanisms, see for example, Bernstein, et al., RNA. 2001, 7(11):1509-21; Nishikura, Cell. 2001, 107(4):415-8 and PCT Publication No. WO 01/36646).

Studies have revealed that siRNA can be effective in vivo in mammals including humans. Specifically, Bitko et al., showed that specific siRNAs directed against the respiratory syncytial virus (RSV) nucleocapsid N gene are effective in treating mice when administered intranasally (Nat. Med. 2005, 11(1):50-55). For reviews of therapeutic applications of siRNAs see for example Batik (Mol. Med 2005, 83: 764-773) and Chakraborty (Current Drug Targets 2007 8(3):469-82). In addition, clinical studies with short siRNAs that target the VEGFR1 receptor in order to treat age-related macular degeneration (AMD) have been conducted in human patients (Kaiser, Am J Ophthalmol. 2006 142(4):660-8). Further information on the use of siRNA as therapeutic agents may be found in Durcan, 2008. Mol. Pharma. 5(4):559-566; Kim & Rossi, 2008. BioTechniques 44:613-616; Grimm & Kay, 2007, JCI, 117(12):3633-41.

The dsRNA as disclosed herein is unmodified, recombinant or chemically modified. Examples of chemical modifications useful in synthesizing dsRNA, including siRNA and siNA are disclosed in PCT Patent Publication No. WO 2009/044392, WO 2011/066475, WO 2011/085056 and are hereby incorporated by reference in its entirety.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. The compositions disclosed herein are administered by any of the conventional routes of administration. It should be noted that the composition can be administered alone or with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles.

In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients disclosed herein and they include liposomes, lipidated glycosaminoglycans and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In general, the active dose of compound for humans is in the range of from lng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24- to 48 hrs or by continuous infusion during a period of 1-2 weeks or longer.

Additionally, provided herein is a method of down regulating expression of a target gene by at least 50% as compared to a control comprising contacting an mRNA transcript of the gene with one or more of the compositions or nucleic acid molecules disclosed herein.

In one embodiment the therapeutic agent inhibits a target gene, whereby the inhibition is selected from the group comprising inhibition of gene function, inhibition of polypeptide and inhibition of mRNA expression.

The pharmaceutical composition is formulated to provide for a single dosage administration or a multi-dosage administration.

In various embodiments the pharmaceutical composition is administered intravenously, intramuscularly, locally, or subcutaneously to the subject.

The pharmaceutical composition disclosed herein can also be used in a method for preventing and/or treating a disease as disclosed herein, whereby the method comprises administering the composition or medicament disclosed herein to a subject in need thereof for treating any of the diseases described herein.

### Diagnostics

The compositions disclosed herein are useful in diagnosing ENDO180 expressing cells in biological samples. The delivery system may include a moiety that is detectable in a normal or diseased cell. The detectable moieties contemplated herein include, but are not limited to, fluorescent, metallic, enzymatic and radioactive markers such as biotin, gold, ferritin, alkaline phosphatase, β-galactosidase, peroxidase, urease, fluorescein, rhodamine, and radioisotopes including tritium, ¹⁴C and iodination.

### Delivery

In some embodiments the compositions disclosed herein are delivered to the target tissue by systemic administration.

The compositions disclosed herein are administered and dosed in accordance with good medical practice, taking into account the clinical condition of the individual patient, the disease to be treated, the site and method of administration, scheduling of administration, patient age, sex, body weight and other factors known to medical practitioners.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

In some embodiments the compositions are “stable” and are not significantly degraded after exposure to serum or cellular proteinases, lipases and or nucleases. A suitable assay for determining stability includes a serum stability assay or a cellular extract assay, known in the art.

“Systemic delivery,” as used herein, refers to delivery that leads to a broad biodistribution of the composition within a subject. Systemic delivery of the compositions disclosed herein can be by any means known in the art including, for example, intravenous, subcutaneous, or intraperitoneal administration. In a preferred embodiment, the composition is delivered systemically by intravenous delivery.

In preferred embodiments the subject being treated is a warm-blooded animal and, in particular, mammals including human.

Suitable methods for delivery of the compositions disclosed herein to an isolated cell include, among others, transfection, lipofection, and electroporation.

### Combination Therapy

In various embodiments, combination therapy is provided. In one embodiment, the co-administration of two or more therapeutic agents achieves a synergistic effect, i.e., a therapeutic affect that is greater than the sum of the therapeutic effects of the individual components of the combination. In another embodiment, the co-administration of two or more therapeutic agents achieves an additive effect.

The active ingredients that comprise a combination therapy may be administered together via a single dosage form or by separate administration of each active agent. In certain embodiments, the first and second therapeutic agents are administered in a single dosage form. Alternatively, the first therapeutic agent and the second therapeutic agents may be administered as separate compositions. The first active agent may be administered at the same time as the second active agent or the first active agent may be administered intermittently with the second active agent. The length of time between administration of the first and second therapeutic agent may be adjusted to achieve the desired therapeutic effect. For example, the second therapeutic agent may be administered only a few minutes (e.g., 1, 2, 5, 10, 30, or 60 min) or several hours (e.g., 2, 4, 6, 10, 12, 24, or 36 hr) after administration of the first therapeutic agent. In certain embodiments, it may be advantageous to administer more than one dosage of one of the therapeutic agents between administrations of the second therapeutic agent. For example, the second therapeutic agent may be administered at 2 hours and then again at 10 hours following administration of the first therapeutic agent. Alternatively, it may be advantageous to administer more than one dosage of the first therapeutic agent between administrations of the second therapeutic agent. Importantly, it is preferred that the therapeutic effects of each active ingredient overlap for at least a portion of the duration of each therapeutic agent so that the overall therapeutic effect of the combination therapy is attributable in part to the combined or synergistic effects of the combination therapy.

Disclosed herein are compositions and the use of compositions useful in targeted delivery of therapeutic cargo and diagnostic cargo to a cell and said compositions may be beneficially employed in the treatment of a subject suffering from a proliferative disease including cancer and fibrotic disease.

### Methods of Treatment

An additional aspect of the disclosure provides for methods of treating a subject suffering from a proliferative disease including cancer, metastatic disease and fibrosis. Methods for therapy of diseases or disorders associated with uncontrolled, pathological cell growth, e.g. cancer and organ fibrosis are provided. In particular, the compositions disclosed herein are useful in treating proliferative diseases in which ENDO180 is expressed in at least a portion of the diseased cells and or tissue. Further provided are methods for treating or preventing the incidence or severity of a disease or condition and/or for reducing the risk or severity of a disease or condition in a subject in need thereof wherein the disease or condition and/or a symptom and/or risk associated therewith is associated with expression of a gene associated with aberrant expression of ENDO180. In a preferred embodiment the subject is a human subject.

### Cancer Therapy

“Cancer” or “Tumor” refers to an abnormal proliferation of cells. These terms include both primary tumors, which may be benign or malignant, as well as secondary tumors, or metastases which have spread to other sites in the body. Examples of proliferative diseases include, inter alia: carcinoma (e.g.: breast, colon and lung), leukemia such as B cell leukemia, lymphoma such as B-cell lymphoma, blastoma such as neuroblastoma and melanoma and sarcoma. It will be acknowledged that the pharmaceutical compositions disclosed herein are used for any disease which involves undesired development or growth of vasculature, including angiogenesis, as well as any of the diseases and conditions described herein, in particular diseases and disorders exhibiting aberrant ENDO180 expression.

Provided herein are methods and compositions for treating a patient suffering from a proliferative disease, including a cancerous proliferative disease (e.g. lung cancer, breast cancer, cervical cancer, colon cancer, gastric cancer, kidney cancer, leukemia, liver cancer, lymphoma, ovarian cancer, pancreatic cancer, prostate cancer, rectal cancer, sarcoma, skin cancer, testicular cancer, and uterine cancer) in which the cancer cell expresses an ENDO180 polypeptide. In one particular embodiment, the cancer is renal cancer including RCC and TCC.

“Cancer and “cancerous disease” are used interchangeably and refer to a disease that is caused by or results in inappropriately high levels of cell division, inappropriately low levels of apoptosis, or both. Examples of cancerous diseases include, without limitation, leukemias (e. g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyo sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, crailiopharyngioma, ependymoma, pinealoma, hemangioblastoma, acoustic neuroma, oligodenroglioma, schwamioma, meningioma, melanoma, neuroblastoma, and retinoblastoma). Treatment of metastases of a primary cancer is included. In some preferred embodiments the compositions are useful in treating renal cancer, breast cancer, ovarian cancer and metastases thereof in various organs including lung and bone.

As used herein, the term “proliferative disease” refers to any disease in which cellular proliferation, either malignant or benign, contributes to the pathology of the condition. Such unwanted proliferation is the hallmark of cancer and many chronic inflammatory diseases, thus examples of “proliferative disease” include the cancers listed supra and chronic inflammatory proliferative diseases such as psoriasis, inflammatory bowel disease and rheumatoid arthritis; proliferative cardiovascular diseases such as restenosis; proliferative ocular disorders such as diabetic retinopathy; and benign hyperproliferative diseases such as hemangiomas.

### Fibrotic Disease

Fibrotic diseases are a group of chronic disease characterized by the excess production of a fibrous material called the extracellular matrix, which contributes to abnormal changes in tissue architecture and interferes with normal organ function. Millions of people worldwide suffer from these chronic diseases, that are often life threatening. Unfortunately, although fibrosis is widely prevalent, debilitating and often life threatening, there is no effective treatment currently available.

The human body responds to trauma and injury by scarring. Fibrosis, a type of disorder characterized by excessive scarring, occurs when the normal wound healing response is disturbed. During fibrosis, the wound healing response continues causing an excessive production and deposition of collagen.

Although fibrotic disorders can be acute or chronic, the disorders share a common characteristic of excessive collagen accumulation and an associated loss of function when normal tissue is replaced with scar tissue.

Fibrosis results from diverse causes, and may be established in various organs. Cirrhosis, pulmonary fibrosis, sarcoidosis, keloids, hypertension and kidney fibrosis, are all chronic diseases that induce a progressive fibrosis which causing a continuous loss of tissue function.

In some embodiments the preferred indications include liver fibrosis and lung fibrosis, for example liver cirrhosis due to Hepatitis C post liver transplant or Non-Alcoholic Steatohepatitis (NASH); Idiopathic Pulmonary Fibrosis; Radiation Pneumonitis leading to Pulmonary Fibrosis; Diabetic Nephropathy; Peritoneal Sclerosis associated with continual ambulatory peritoneal dialysis (CAPD) and Ocular cicatricial pemphigoid. Acute fibrosis (usually with a sudden and severe onset and of short duration) occurs as a common response to various forms of trauma including accidental injuries (particularly injuries to the spine and central nervous system), infections, surgery (cardiac scarring following heart attack), burns, environmental pollutants, alcohol and other types of toxins, acute respiratory distress syndrome, and radiation and chemotherapy treatments. All tissues damaged by trauma are prone to scar and become fibrotic, particularly if the damage is repeated. Deep organ fibrosis is often extremely serious because the progressive loss of organ function leads to morbidity, hospitalization, dialysis, disability and even death. Fibrotic diseases or diseases in which fibrosis is evident include pulmonary fibrosis, interstitial lung disease, human fibrotic lung disease, liver fibrosis, cardiac fibrosis, macular degeneration, retinal and vitreal retinopathy, myocardial fibrosis, Grave's ophthalmopathy, drug induced ergotism, cardiovascular disease, atherosclerosis/restenosis, keloids and hypertrophic scars, Hansen's disease and inflammatory bowel disease, including collagenous colitis.

Further information on different types of fibrosis may be found for example in Yu et al (2002), “Therapeutic strategies to halt renal fibrosis”, Curr Opin Pharmacol. 2(2):177-81; Keane and Lyle (2003), “Recent advances in management of type 2 diabetes and nephropathy: lessons from the RENAAL study”, Am J Kidney Dis. 41(3 Suppl 2): S22-5; Bohle et al (1989), “The pathogenesis of chronic renal failure”, Pathol Res Pract. 185(4):421-40; Kikkawa et al (1997), “Mechanism of the progression of diabetic nephropathy to renal failure”, Kidney Int Suppl. 62:S39-40; Bataller and Brenner (2001), “Hepatic stellate cells as a target for the treatment of liver fibrosis”, Semin Liver Dis. 21(3):437-51; Gross and Hunninghake (2001) “Idiopathic pulmonary fibrosis”, N Engl J Med. 345(7):517-25; Frohlich (2001) “Fibrosis and ischemia: the real risks in hypertensive heart disease”, Am J Hypertens; 14(6 Pt 2):194S-199S.

### Diabetic Nephropathy

Diabetic nephropathy, hallmarks of which are glomerulosclerosis and kidney fibrosis, is the single most prevalent cause of end-stage renal disease in the modern world, and diabetic patients constitute the largest population on dialysis. Such therapy is costly and far from optimal. Transplantation offers a better outcome but suffers from a severe shortage of donors. More targeted therapies against diabetic nephropathy (as well as against other types of kidney pathologies) are not developed, since molecular mechanisms underlying these pathologies are largely unknown. Identification of an essential functional target gene that is modulated in the disease and affects the severity of the outcome of diabetes nephropathy has a high diagnostic as well as therapeutic value.

It is known in the art that many pathological processes in the kidney eventually culminate in similar or identical morphological changes, namely glomerulosclerosis and fibrosis. Human kidney disease may evolve from various origins including glomerular nephritis, nephritis associated with systemic lupus, cancer, physical obstructions, toxins, metabolic disease and immunological diseases, all of which culminate in kidney fibrosis. The meaning of this phenomenon is that different types of insults converge on the same single genetic program resulting in two hallmarks of fibrosis: the proliferation of fibroblasts and overproduction by them of various protein components of connective tissue. In addition, thickening of the basal membrane in the glomeruli accompanies interstitial fibrosis and culminates in glomerulosclerosis. Without wishing to be bound to theory, genes encoding proteins that are involved in kidney fibrosis and glomerulosclerosis may be roughly divided into two groups:

1. Genes, the expression of which triggers proliferation of fibroblasts and their overproduction of various protein components of connective tissue. These may be specific to different pathological conditions; and

2. Genes, the expression of which leads to the execution of the “fibrotic or sclerotic programs”. These may be common to all renal pathologies leading to fibrosis and glomerulosclerosis.

The identification of genes that belong to the second group should contribute to the understanding of molecular mechanisms that accompany fibroblast and mesangial cell proliferation and hypersecretion, and may constitute genetic targets for drug development, aimed at preventing renal failure. Application of such drugs is expected to suppress, retard, prevent, inhibit or attenuate progression of fibrosis and glomerulosclerosis.

### Kits

Kits comprising all or part of the compositions are further provided. A “kit” refers to any manufacture (e.g., a package or a container) comprising the composition or components of the composition. The kit may be used for performing the methods disclosed herein, including therapeutic treatment and diagnostics. Additionally, the kit may contain a package insert describing the kit, its content and methods for use.

The invention has been described in an illustrative manner, and it is to be understood that the terminology which has been used is intended to be in the nature of words of description rather than of limitation.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ (In cell) PCR in combination with Flow Cytometry can be used for detection of cells containing specific DNA and mRNA sequences (Testoni et al., 1996, Blood 87:3822.)

General methods in immunology: Standard methods in immunology known in the art and not specifically described are generally followed as in Stites et al (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays: ELISA immunoassays are well known to those skilled in the art. Both polyclonal and monoclonal antibodies can be used in the assays. Where appropriate, other immunoassays such as radioimmunoassays (RIA) can be used as are known to those skilled in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al., Molecular Cloning: A Laboratory Manual, Cold Springs Harbor, New York, 1989.

### Antibody Production

By the term “antibody” as used herein is meant both polyclonal and monoclonal complete antibodies as well as fragments thereof, such as Fab, F(ab′)2, scFv and Fv, which are capable of binding the epitope determinant. These antibody fragments retain the ability to selectively bind with its antigen or receptor and are exemplified as follows, inter alia:

A Fab, the fragment which contains a monovalent antigen-binding fragment of an antibody molecule can be produced by digestion of whole antibody with the enzyme papain to yield a light chain and a portion of the heavy chain;

A (Fab′)2, the fragment of the antibody that can be obtained by treating whole antibody with the enzyme pepsin without subsequent reduction; F(ab′2) is a dimer of two Fab fragments held together by two disulfide bonds;

A Fv, defined as a genetically engineered fragment containing the variable region of the light chain and the variable region of the heavy chain expressed as two chains; and

A scFv fragment (i.e. a single chain antibody (“SCA”), defined as a genetically engineered molecule containing the variable region of the light chain and the variable region of the heavy chain linked by a suitable polypeptide linker as a genetically fused single chain molecule.

Such fragments having antibody functional activity can be prepared by methods known to those skilled in the art (Bird et al. (1988) Science 242:423-426). (Mab or mAb is used herein as abbreviations for monoclonal antibody. MB is used herein as an abbreviation for minibody.)

Conveniently, antibodies may be prepared against an immunogen or portion thereof, for example, a synthetic peptide based on the sequence, or prepared recombinantly by cloning techniques or the natural gene product and/or portions thereof may be isolated and used as the immunogen Immunogens can be used to produce antibodies by standard antibody production technology well known to those skilled in the art, as described generally in Harlow and Lane (1988), Antibodies: A Laboratory Manual, Cold Spring Harbor Laboratory, Cold Spring Harbor, N.Y., and Borrebaeck (1992), Antibody Engineering—A Practical Guide, W.H. Freeman and Co., NY.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific; that is, the sera can be absorbed against related immunogens so that no cross-reactive antibodies remain in the sera, rendering it monospecific.

For producing monoclonal antibodies the technique involves hyperimmunization of an appropriate donor with the immunogen, generally a mouse, and isolation of splenic antibody-producing cells. These cells are fused to an immortal cell, such as a myeloma cell, to provide a fused cell hybrid that is immortal and secretes the required antibody. The cells are then cultured, in bulk, and the monoclonal antibodies harvested from the culture media for use.

For producing recombinant antibody see generally Huston et al. (1991) “Protein engineering of single-chain Fv analogs and fusion proteins” in Methods in Enzymology (JJ Langone, ed., Academic Press, New York, N.Y.) 203:46-88; Johnson and Bird (1991) “Construction of single-chain Fvb derivatives of monoclonal antibodies and their production in Escherichia coli in Methods in Enzymology (J J Langone, ed.; Academic Press, New York, N.Y.) 203:88-99; Mernaugh and Mernaugh (1995) “An overview of phage-displayed recombinant antibodies” in Molecular Methods In Plant Pathology (RP Singh and US Singh, eds.; CRC Press Inc., Boca Raton, Fla.: 359-365). Additionally, messenger RNAs from antibody-producing B-lymphocytes of animals, or hybridomas can be reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe (1982), Immunochemistry in Practice, Blackwell Scientific Publications, Oxford). The binding of antibodies to a solid support substrate is also well known in the art (for a general discussion, see Harlow & Lane (1988) Antibodies: A Laboratory Manual, Cold Spring Harbor Laboratory Publications, New York; and Borrebaeck (1992), Antibody Engineering—A Practical Guide, W.H. Freeman and Co.). The detectable moieties or label contemplated herein include, but are not limited to, fluorescent, metallic, enzymatic and radioactive markers such as biotin, gold, ferritin, alkaline phosphatase, β-galactosidase, peroxidase, urease, fluorescein, rhodamine, tritium, 14C and iodine.

Recombinant Protein Purification: For standard purification, see Marshak et al. (1996), “Strategies for Protein Purification and Characterization. A laboratory course manual.” CSHL Press.

### Example 1

### Anti-ENDO180 Antibodies

ENDO180 is also known as the C-type mannose receptor 2 precursor. The polynucleotide sequence of human ENDO180 mRNA is set forth in accession number NM_{—}006039.3: 5983 bases, of that the open reading frame (ORF) is 4439 bases (from 117-4441); the polypeptide sequence of 1479 amino acids (aa) is set forth in accession number NP_{—}006030 with gene identifier number: GI:110624774. The mouse mRNA sequence is 5818 bases, accession number MMU56734 with ORF of 1479 aa.

ENDO180 comprises several protein domains, as follows: 1-31 aa SP (signal peptide); 41-161 aa cysteine rich N-terminal domain; 180-228 aa FNII (fibronectin type II) domain; 8 CDR (carbohydrate recognition domain) domains 1CRD-8CRD (235-360 aa 1CRD, 382-505 aa 2CRD, 521-645 aa 3CRD, 669-809 aa 4CRD, 825-951 aa 5CRD, 972-1108 aa 6CRD, 1161-1244 aa 7CRD, 1261-1394 aa 8CRD); 1413-1435 aa 1 TM (transmembrane domain), 1437-1479 aa-cytoplasmic domain. In some embodiments the ENDO180 polypeptide is substantially identical to an amino acid sequence set forth in SEQ ID NO:2, (NCBI identifier: gi|110624774|ref|NP_{—}006030.2|) encoded by a polynucleotide substantially identical to a nucleic acid sequence set forth in SEQ ID NO:1 (NCBI identifier: gi|110624773|ref|NM_{—}006039.3|).

Provided below are polynucleotide and amino acid sequences disclosed herein: polynucleotide sequence of extracellular domain of human ENDO180 (amino acids 1-522) with FLAG sequence, (pcDNA3-5′hENDO180-FLAG construct, SEQ ID NO:3); polypeptide sequence of SEQ ID NO:3 (SEQ ID NO:4); polynucleotide sequence of scFv clone G7V (SEQ ID NO:5); polypeptide sequence of scFv clone G7V (SEQ ID NO:6; also known as minibody or “MB”); heavy chain CDR3 of G7V (SEQ ID NO:7); light chain CDR3 of G7V (SEQ ID NO:8).

The antibody produced from hybridoma cell line E3-8D8 (also known herein as 8D8 or e3b3 or 8d8e3b3; deposited in BCCM under Accession Number LMBP 7203CB) and the recombinant anti-ENDO180 antibodies disclosed herein are described in PCT patent publication WO2010/111198, hereby incorporated by reference in its entirety. In some embodiments the preferred ENDO180 targeting agent is selected from
a. an isolated monoclonal antibody or an antigen-binding fragment thereof, produced by the hybridoma cell line E3-8D8 deposited with the BCCM Accession Number under LMBP 7203CB;
b. an antibody or an antigen-binding fragment thereof that binds to the same epitope as the antibody of (a);
c. a humanized version of the antibody or an antigen-binding fragment thereof, of (a) or a humanized version of the antibody or antigen-binding fragment of (b);
d. a chimeric version of the antibody or an antigen-binding fragment thereof, of (a) or a chimeric version of the antibody or antigen-binding fragment of (b);
e. a recombinant polypeptide comprising the antigen-binding domain of the antibody in (a) or antigen-binding fragment thereof which is internalized in to a cell by the ENDO180 receptor;
f. an antigen-binding fragment of an antibody comprising a polypeptide substantially similar to SEQ ID NO:6; and
g. a recombinant polypeptide comprising the CDRs having an amino acid sequence substantially similar to amino acid sequences set forth in SEQ ID NO:7 and 8.

### Example 2

### Lipid Compositions

Objective: The main objective of this study was to develop a platform to selectively deliver cargo, including small molecules and oligonucleotides, such as antisense and dsRNA to target cells. Specifically, the cargo was delivered to cells expressing the endocytic ENDO180 receptor that is overexpressed on activated myofibroblasts in fibrotic tissues and tumors, on an invasive subset of tumor cells and especially on sarcomas and on neovasculature endothelium.

Specificity of cellular uptake of lipid-based nanoparticles (“lipid particles”, “lipid NPs”) decorated with anti-ENDO180 antibodies was achieved using NRK52 (also known as NRK) cell line stably transfected to express ENDO180 (NRK-ENDO or NRK-ENDO180). As a control, the NRK52 cell line stably transfected with the pIRESPuro empty vector was used.

Materials and Methods:

### Compositions and Physicochemical Characterization:

Compositions comprising lipid and an ENDO180 targeting moiety for targeted delivery of therapeutic or diagnostic cargo were developed as follows:

1—lipid particles carrying a small molecule (for example a cancer therapeutic including doxorubicin or mitomycin as a small hydrophilic model drug);

2—lipid particles carrying dsRNA (for example Cy3-siRNAs as model dsRNA).

Cy3 labeled siRNA includes an antisense strand with unmodified ribonucleotides in positions 2, 4, 6, 8, 10, 12, 14, 16 and 18, and 2′O-Methyl sugar modified ribonucleotides in positions 1, 3, 5, 7, 9, 11, 13, 15, 17, and 19, and a Cy3 moiety covalently attached to the 3′ terminus of the antisense strand; and a sense strand with unmodified ribonucleotides in positions 1, 3, 5, 7, 9, 11, 13, 15, 17, and 19 and 2′O-Methyl sugar modified ribonucleotides in positions 2, 4, 6, 8, 10, 12, 14, 16 and 18. Materials: High-purity hydrogenated soy phosphatidylcholine (HSPC), Cholesterol (Chol) Dioleoyl Phosphatidylethanolamine (DOPE) were purchased from Avanti Polar lipids Inc., (Alabaster, Ala., USA). Soy phosphatidylcholine (soy-PC) and 1,2-Bis(diphenylphosphino)ethane (DPPE) were purchased from Avanti polar lipids, (Alabaster, Ala., USA). NHS-PEG-DSPE [3-(N-succinimidyloxyglutaryl)aminopropyl, polyethyleneglycol-carbamyl distearoylphosphatidyl-ethanolamine] from NOF cooperation, Tokyo, Japan. Hyaluronan high molecular weight from Genzyme Cooperation (Cambridge, Mass., USA). Cell culture plates and dishes were from Corning Glass Works (New York, N.Y., USA). Polycarbonate membranes were from Nucleopore (Pleasanton, Calif., USA). Total RNAs were extracted with the RNeasy® mini kit from Qiagen, (Valencia, Calif., USA) and reverse-transcribed by Superscript III from Invitrogen (Carlsbad, Calif., USA). Primers for quantitative RT-PCR were obtained from Syntheza, Inc. (Rehovot, Israel). Doxorubicin and mitomycin were purchased from Sigma-Aldrich Co. (St. Louis, Mo., USA). All other reagents were of analytical grade.

Fluorochrome Labeling of 8D8 mAb with Alexa Fluor 488.

1 mg of the E3-8D8 monoclonal antibody (8D8) was used for labeling, using the Alexa Fluor 488 and 647 Protein Labeling kits (Invitrogen cat# A10235). The labeling procedure was performed according to manufacturer's instructions and purified on a desalting column to separate non-bound dye.

Composition 1. Lipid-based nanoparticle preparation—PEG-spacer, coated with anti-ENDO180 antibody and carrying doxorubicin as therapeutic agent (cargo).

Composition 1 comprises hydrogenated soybean phosphatidylcholine (HSPC), cholesterol (Chol), dioleoyl phosphatidylethanolamine (DOPE) and NHS-PEG-DSPE [3-(N-succinimidyloxyglutaryl)aminopropyl, polyethyleneglycol-carbamyldistearoylphosphatidyl-ethanolamine] (NOF cooperation, Tokyo) at molar ratios of about 75:20:4.5:0.5 (HSPC:chol:DOPE:NHS-PEG-DSPE).

Briefly, multilamellar vesicles (MLV) were prepared by a lipid-film method and evaporated to dryness using a buchi-rotovap (Peer and Margalit, 2000, Arch Biochem Biophy 383(2):185-90; Peer and Margalit, 2004, Neoplasia 6(4):343-53; Peer et al., 2008, Science 319(5863):627-630). The lipid film was hydrated with doxorubicin resuspended in saline at pH of 7.4 to create MLV. Lipid mass was measured as previously described (Peer et al, 2008). Resulting MLV were extruded into small unilamellar nano-scale vesicles (SUV) with a Thermobarrel Lipex extruder (Lipex Biomembranes Inc., Vancouver, British Columbia, Canada) at 60° C. under nitrogen pressure of 300 to 550 psi. The extrusion was carried out in a stepwise manner using progressively decreasing pore-sized membranes (from 1, 0.8, 0.6, 0.4, 0.2, to 0.1 μm) (Nucleopore, Whatman), with 10 cycles per pore-size.

### Surface Modification and Purification of Anti-ENDO180-Coated Lipid-Nanoparticles and Isotype Control Particles

Anti-ENDO180 (clone 8D8) or Isotype control (non-binding mouse IgG2a) antibodies were concentrated using Amicon Tube (MW cut off of 100KDa) to a final concentration of 10 mg/mL as determine by IgG absorbance at 280 nm using a NanoDrop 1000 spectrophotometer (Thermo Scientific).

Covalent association of antibody to lipid was performed with EDC-NHS crosslinkers. at room temperature overnight in PBS in 1mL reaction vials that include 504 of mAb (10 mg/mL) and lipid particle at 10 mg/mL at 9504.

Purification of excessive 8D8 mAb was made using a Sepharose CL-4B column equilibrated with HEPES buffer-saline at pH 7.4.

Doxorubicin (DOX) was quantified by fluorescence with a calibration curve freshly made for each experiment.

Composition 2. Lipid-Based Nanoparticle Preparation—Hyaluronan Spacer Coated with Anti-ENDO180 Antibody and Carrying Labeled siRNA

Multilamellar vesicles (MLV) comprising Dioleoyl Phosphatidylethanolamine (DOPE), 1,2-di-O-octadecenyl-3-trimethylammonium propane (DOTMA) and cholesterol (Chol) all from Avanti Polar Lipids, Inc., (Alabaster, Ala., USA) at molar ratios of about 4:2:1 (DOPE:DOTMA:Chol), were prepared by a lipid-film method (Peer and Margalit 2004). The lipid film was hydrated with Cy3-labeled siRNA suspended in DEPC-water to create MLV.

Effcacy of siRNA encapsulation: Cargo loading was performed accoring to methods disclosed in Landesman-Milo, et al., (2012, Cancer Lett. pii: S0304-3835 (12)00512-5), incorporated herein by reference in its entirety. Briefly, siRNA encapsulation efficiency was determined by the Quant-iT™ RiboGreen® RNA Assay Kit (Invitrogen) and was performed by comparing fluorescence of the RNA binding dye RiboGreen in the LNP (lipid nanoparticles) and HA-LNP (hyaluronic-bound lipid nanoparticles) samples, in the presence and absence of detergent. In the untreated samples, fluorescence is measured from unencapsulated siRNA (free siRNA) while in the detergent treated samples the fluorescence is measured from total siRNA. The percent encapsulation is calculated sas follows:

% siRNA encapsulation=[1−(free siRNA conc./total siRNA conc.)]×100.

Lipid mass was measured as previously described (Peer et al., 2008). Resulting MLV were extruded into unilamellar nano-scale vesicles (ULV) with a Thermobarrel Lipex extruder (Lipex Biomembranes Inc., Vancouver, British Columbia, Canada) at room temperature under nitrogen pressure of 300 to 550 psi. The extrusion was carried out in a stepwise manner using progressively decreasing pore-sized membranes (from 1, 0.8, 0.6, 0.4, 0.2, to 0.1 μm) (Nucleopore, Whatman), with 10 cycles per pore-size.

### Surface Modification and Purification of Anti-ENDO180-Coated Lipid-Nanoparticles and Isotype Control Particles.

ULV were coated with high-molecular weight hyaluronan (HA) which stabilizes the particles and serves as a scaffold for mAb binding (Peer et al., 2008). Briefly, HA was dissolved in water and pre-activated with EDC, at pH 4.0 for 2 h at 37° C. Resulting activated HA was added to a suspension of DOPE-containing ULV in 0.1 M borate buffer pH 8.6, and incubated overnight at 37° C., with gentle stirring. Resulting HA-ULV were separated by centrifugation (1.3×105 g, 40 C, for 1 h) and washed four times. The final HA/lipid ratio was typically 57-70 μg HA/gmole lipid as assayed by 3H-HA (ARC, Saint Louis, Mich.).

HA-modified nanoparticles (NPs) were coupled to the anti-ENDO180 or anti-IgG mAbs using an amine-coupling method. Briefly, 50 μL HA-modified lipid particles (40 mg/mL) were incubated with 200 μL of 400 mmol/L 1-(3-dimethylaminopropyl)-3-ethylcarbodimide hydrochloride (EDAC, Sigma-Aldrich, Saint Louis, Mich.) and 200 μL of 100 mmol/L-N-hydroxysuccinimide (NHS, Fluka, Sigma-Aldrich, Saint Louis, Mich.) for 20 minutes at room temperature with gentle stirring. Resulting NHS-activated HA-NPs were mixed with 50 μL mAb (10 mg/mL in HBS, pH 7.4 of Anti-ENDO180 clone 8D8 or its isotype control, mouse IgG2a) and incubated overnight at room temperature with gentle stirring. Twenty microliter 1 M ethanolamine HCl (pH 8.5) was then added to block reactive residues. The resulting immuno-NPs were purified using a size exclusion column packed with Sepharose CL-4B beads (Sigma-Aldrich, Saint Louis, Mich.) and equilibrated with HBS, pH 7.4 to remove unattached mAbs.

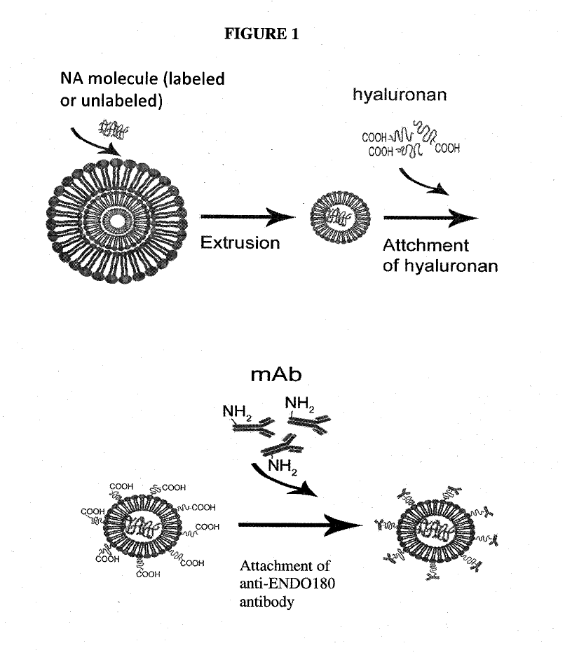

FIG. 1 shows a scheme for the generation of the HA coated lipid particles.

Composition 3. Lipid-Based Nanoparticle Preparation-Hyaluronan Spacer, Coated with Anti-ENDO180 Antibody

Multilamellar vesicles comprising 60% soy phosphatidylcholine (soy-PC), 20% DPPE, and 20% cholesterol (mol/mol) at a concentration of 40 mg/ml (soy PC-273 mg, DPPE-81.2 mg, cholesterol-145.4 mg in 10 ml of ethanol) were prepared by a lipid-film method and evaporated to dryness in a rotary evaporator (BUCHI R-210), as described above. Following the evaporation, the dry lipid film was hydrated in 10 ml of HBS (150 mM NaCl, 20 mM Hepes) (pH 7.4) and the solution was shaken (2 hr 65° C.) to create MLV. Lipid mass was measured as previously described (Peer et al., 2008). The resulting MLV were extruded into unilamellar nano-scale vesicles (ULV) with an average size of ˜150nm (Zetasizer Nano ZS system) with a Thermobarrel Lipex extruder (Lipex Biomembranes Inc., Vancouver, British Columbia, Canada), as described above.

### Surface Modification and Purification of Anti-ENDO180-Coated Lipid-Nanoparticles and Isotype Control Particles.

High-molecular weight hyaluronan (HA) (700 Kda Lifecore) was dissolved in 0.2M MES buffer (pH 5.5) to a final concentration of 5 mg/ml, and activated with EDC and sulfo-NHS at a molar ratio of 1:1:6. After 30 min of activation the ULV were added and the pH was adjusted to 7.4. The solution was incubated at room temp (2 hr). The free HA was removed by 3 ultracentrifugation cycles. The resulting HA-ULVs had an average size of 130 nm.

mAb Binding and Purification

Anti-ENDO180 8D8 mAbs were concentrated to a final concentration of 10 mg/ml (Centricon Centrifugal Filter units). 20 μl of antibodies were activated with 1.2 μg of EDC and 1.44 μgr of sulfo-NHS (pH 5.5). After incubation at room temperature for 30 min, 0.8 mg of lipid particles were added and the pH adjusted to pH7.4. The lipid particles were incubated overnight at 4° C. Lipid particles and free antibodies were separated on a CL-4B column.

### Example 3

### Analysis of Compositions

Fluorescence Activated Cell Sorting (FACS) studies.

For binding analysis, 3.5×10⁵ cells were trypsinized, spun down, re-suspended with FACS buffer (1% fetal bovine serum in 1×PBS), incubated for 30 minutes on ice with anti ENDO180 mAbs (1 μg) and washed with 1 ml FACS buffer. mAb samples were incubated with a secondary FITC conjugated goat anti mouse IgG antibody (115-095-072) (1:100, 150 μg/ml) in 50 μl of FACS buffer 30 minutes on ice, resuspended in 1 ml FACS buffer and analyzed using a FACS Calibur flow cytometer.

### Particle Size Distribution and Zeta Potential Measurements.

Particle size distribution and mean diameter of NPs, or 8D8-coated NPs were measured on a Malvern Zetasizer Nano ZS zeta potential and dynamic light scattering instrument (Malvern Instruments, Southborough, Mass.) using the automatic algorithm mode and analyzed with the PCS 1.32a. All measurements were done in 0.01 mol/l NaCl, pH 6.7, at room temperature.

### Binding of 8D8-Coated NPs to Cells.

About 0.5×10⁶ ENDO180-expressing NRK52 cells (NRK-ENDO180) were collected per FACS tube, in 1mL DMEM media spun down and re-suspended in 1mL FACS buffer (99% PBS+1% FCS). Cells were spun down. Supernatant was discarded and the pellet was resuspended with Alexa 488-labeled-8D8-coated NPs or IgG-NPs, (at 1:25-1:75 dilution corresponding to 10-30 μg/mL) and incubated at 4° C. for 30 min. 1mL FACS buffer was added, and cells were spun down. Supernatant was discarded. Then, cells were resuspended in 200 uL FACS buffer (for immediate analysis). Flow cytometry analysis was performed on a FACScan (BD Biosciences, San Jose, Calif., USA) and analyzed using flowjo software (Tree Star Inc., Ashland, Oreg., USA).

### Confocal Microscopy Analysis

In order to detect siRNA delivery in cells, Cy5-labeled siRNA entrapped in the 8D8-coated NPs (Composition 2) were used. A comprehensive confocal analysis was made using the Scanning module of Zeiss LSM 510 META.

The unique scanning module is the core of the LSM 510 META. It contains motorized collimators, scanning mirrors, individually adjustable and positionable pinholes, and highly sensitive detectors including the META detector. All these components are arranged to ensure optimum specimen illumination and efficient collection of reflected or emitted light. A highly efficient optical grating provides an innovative way of separating the fluorescence emissions in the META detector. The grating projects the entire fluorescence spectrum onto the 32 channels of the META detector. Thus, the spectral signature is acquired for each pixel of the scanned image and subsequently can be used for the digital separation into component dyes.

### Internalization Studies

Internalization assays were performed in 24 well plates. 1×10⁵ A549 or NRK-ENDO180 or NRK naive cells were seeded on cover slips in RPMI or DMEM medium respectively, supplemented with antibiotics, L-Glutamine and 10% fetal calf serum (Biological Industries, Beit Haemek, Israel).

For membrane staining, cells were stained with CellTracker™ DilC18 (5)-DS solution (Invitrogen, Carlsbad, Calif., USA), diluted 1:5000 with PBS. For cell membrane labeling, Concanavalin A, Alexa fluor 647 conjugate (10 μg/ml) (C21421, Invitrogen) was used. For nuclei staining, cells were stained with Hoechst (1:10,000 in PBS) (33258, Sigma). Cells were exposed to either lipid particles of composition 3 conjugated to anti-ENDO180 mAb (50 μl from stock, according to preparation method) or lipid particles of composition 3 alone (50 μl from the prepared liposomal stock solution) in medium without serum for a period of 1 hour at 37° C. in a humidified atmosphere with 5% CO₂. Subsequently, the cells were washed twice using cold PBS, fixated with 4% paraformaldehyde (PFA) and washed again with cold PBS. Membrane and nuclei staining were performed after fixation.

The cells were mounted using fluorescent mounting medium (Golden Bridge international, Mukilteo, Wash., USA) and fluorescence was measured using Andor Spinning disc confocal microscope and the Meta 510 Zeiss LSM confocal microscope. Laser beams at 405, 488, 561 and 650 nm were used for UV, Rhodamine, Concavaline A and CellTracker™, fluorophores excitation respectively. Serial optical sections of the cells were recorded for each treatment and the images were processed using Zeiss LSM Image browser software.

Selective Killing of NRK Cells Expressing ENDO180 with Doxorubicin Entrapped in 8D8-NPs.

In order to examine the specificity of the targeted delivery system and the ability to selectively deliver a small molecule entity, DOX was entrapped in 8D8-NPs or in IgG-NPs as detailed in the experimental section above. Cells expressing the ENDO180 receptor (NRK-ENDO180+ cells) and cells lacking the receptor (NRK-ENDO180−/− cells) were incubated in 0.5 μM free DOX or DOX entrapped in 8D8-NPs or in IgG-NPs (at the same concentration) for 0.5 h at 37° C. (at a humidified atmosphere with 5% CO₂). Then, the cells were washed and incubated with drug-free media for an additional 72 h (at 37° C. in the incubator) following by the XTT assay.

### Results

### Structural and Physicochemical Characterization of the Lipid Compositions.

Table 1 shows the diameter and surface charge properties of compositions 1 and 2 in all mAb-conjugated NPs.

TABLE 1

| | Size (d. nm) | Zeta potential (mV) |
|---|---|---|
| Type | (average ± SD) | (average ± SD) |
| Uncoated | 138.9 ± 1.115 | −8.88 ± 0.4 |
| X 1HA | 131 ± 1.424 | −19.2 ± 0.757 |
| X 3HA | 136.1 ± 0.5568 | −28.3 ± 0.3 |
| X 6HA | 136.3 ± 0.3606 | −35.7 ± 1.51 |

The data, which are presented here show an average±SD of 3 independent batches for the PC: DPPE:Cholesterol (at a molar ratio of about 3:1:1) lipid nanoparticles. The terms X1 HA, X3 HA and X6 HA refer to the amount of HA bound to the lipid nanoparticles, as a function of the EDC and Sulfo-NHS cross linker concentrations. The HA concentration was 5 mg/ml in each of the formulated HA-NPs. The concentrations of the EDC and Sulfo-NHS cross linker in X1 HA: EDC-7.24 mM; Sulfo-NHS-6 mM (final concentration; in X3 HA: EDC-21 mM; Sulfo-NHS-17.6 mM (final concentration); and in X6 HA:EDC-40.8 mM; SulfoNHS-34 mM (final concentration).

The range of zeta potential of the X1HA, X3HA or X6HA NPs are as follows: X1HA: −20-(−30 mV); X3HA: −28-(−40 mV) and X6HA: −35-(−60 mV).

The size distribution of each type of particle is narrow, and the surface charge is negative. It has recently been demonstrated that while cationic lipid based NPs can induce an immune activation via TLR4, negatively and neutrally charged particles will not (Kedmi et al. 2010, Biomaterials 31(26):6867-75; Kedmi and Peer 2009, Nanomed 4(8):853-5).

### Binding Capacity Screening of Different ENDO180 Expressing Cell Lines to the Different Anti ENDO180 Abs

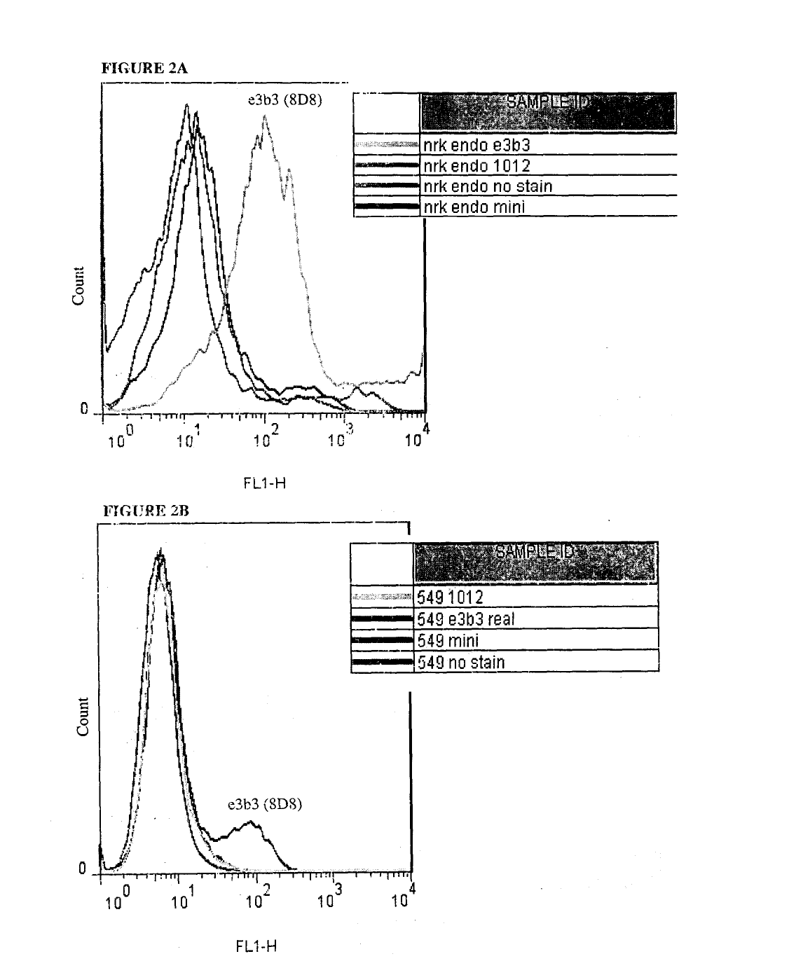

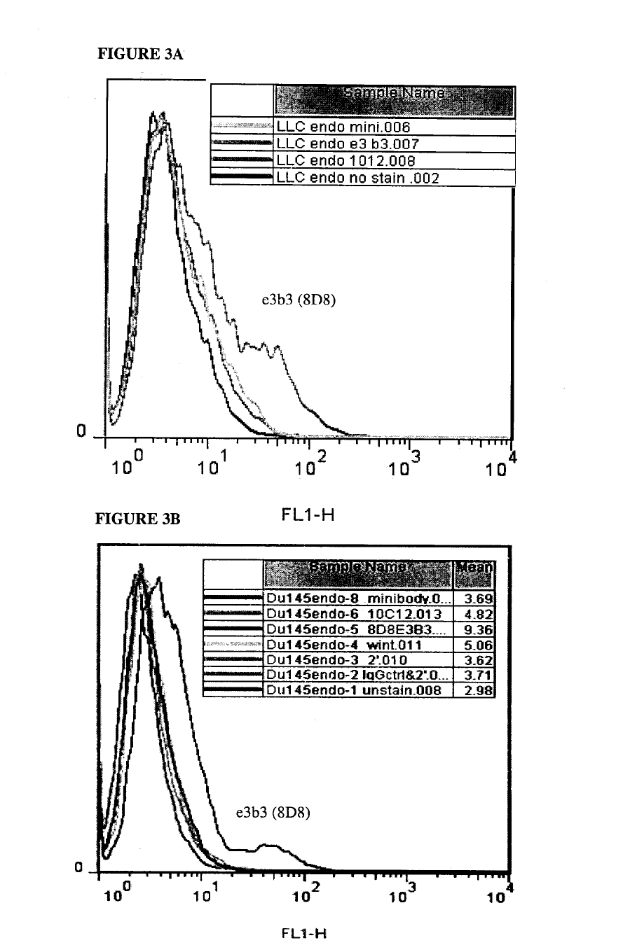

Cell lines which express different ENDO180 receptor levels were tested: NRK+ (a normal rat kidney), DU145⁺ (a human prostate adenocarcinoma), LLC⁺ (a mouse Lewis lung carcinoma), DU145⁻ and LLC⁻ (control cell lines, which are low expressors of ENDO180 receptor levels, i.e. express the PIRES Puro empty plasmid), A549 (human lung carcinoma) and CT26 (mouse colon carcinoma). NRK+, DU145⁺ and LLC⁺ stably express ENDO180 receptor levels. A549 and CT26 express naturally unknown levels of ENDO180 receptor. The binding capacity of the above cell lines was compared using 4 different Abs: mAb 8D8 clone, mAb10C12 clone, minibody (MB) and anti-wnt minibody (negative control). The best binding effect was observed with the NRK-ENDO180; 8D8 mAb pair (FIG. 2A). Significant binding effects, though not as strong as the NRK-ENDO180, were also observed in A549; 8D8 (FIG. 2B) and LLC; ENDO180 pairs (FIG. 3A). A weak binding effect was observed with Du145-ENDO180; 8D8 pair (FIG. 3B).

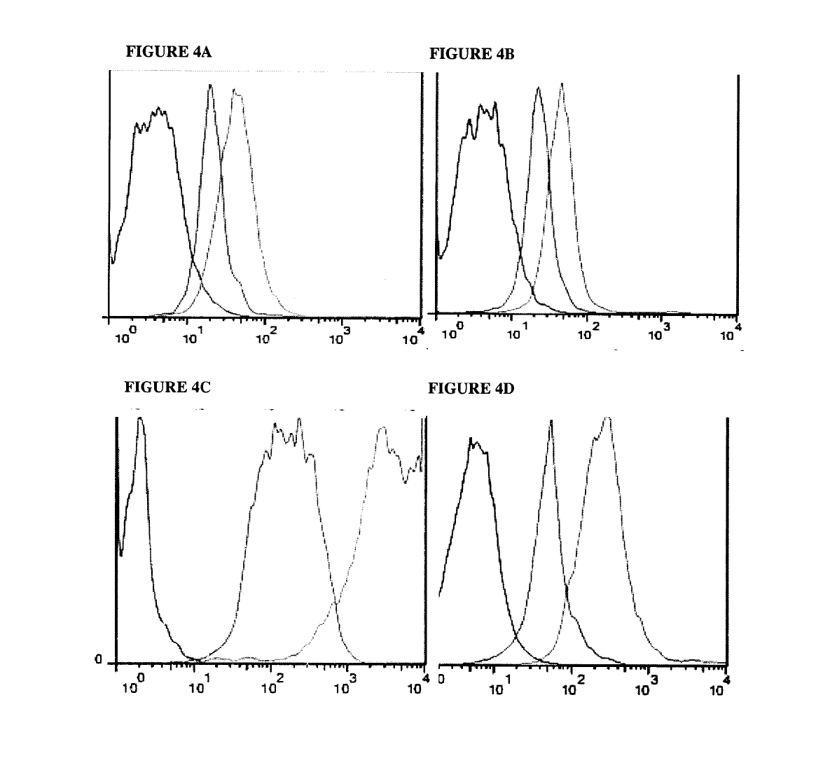

The MB showed a weak binding capacity with all of the tested cell lines. A new batch was tested and the secondary Ab was changed (FITC conjugated goat anti mouse IgG F(ab)2 fragment, 115-095-072, Jackson Immunoresearch). The new MB batch was labeled directly with protein labeling kit. No significant improvement in binding capacity was observed (FIGS. 4A-D). An additional set of binding experiments was performed using Alexa 488 conjugated first mAb (clone 8D8), which showed similar binding results to those obtained with the first unconjugated mAbs (In all scans 4A-4D: right peak:8D8, center peak: minibody, left peak: control unstained cells.

Comparison of Internalization of the Different Anti-ENDO180 Antibodies into Different Cell Lines

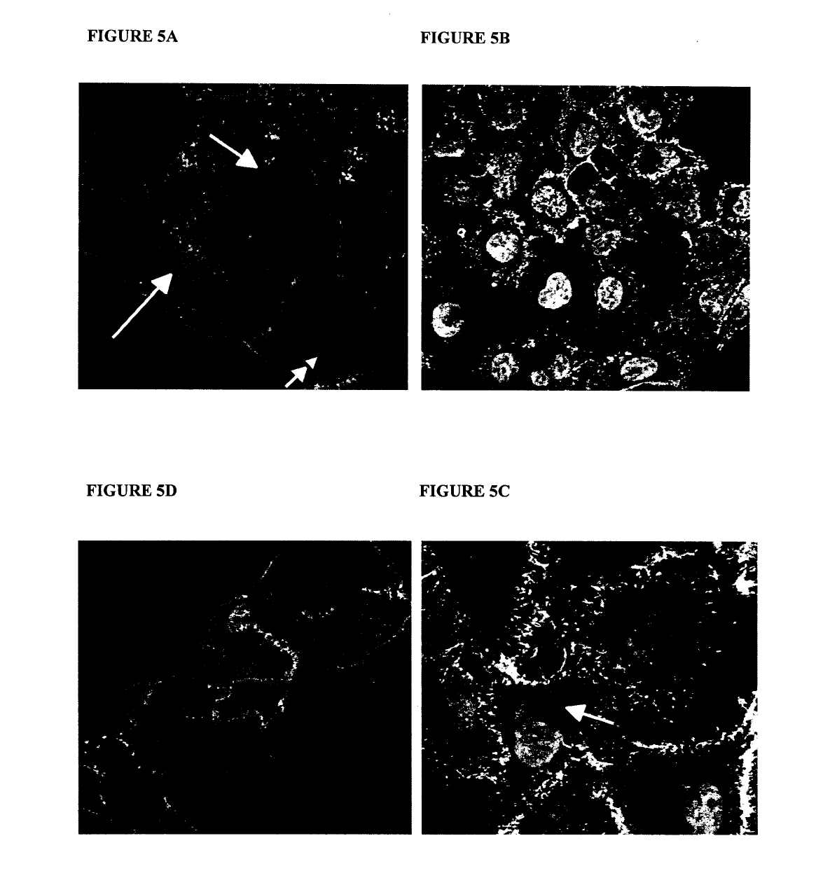

To identify the ENDO180 Abs which best internalize into the above cell lines, internalization tests were performed with the different antibodies and each of the six different cell lines using META 510 LSM confocal microscope. According to the first set of experiments (cells first exposed to unconjugated Abs followed by a secondary FITC goat anti mouse Ab), the best internalization effects were observed with the following: NRK-ENDO180 cells; 8D8 mAb-A549 cells; and DU145-ENDO180; 10C12 mAb-DU145 cell line pairs. However no internalization of MB was observed into the tested cell lines. In addition, both MB and mAb 8D8 were labeled with Alexa Fluor 488, using protein labeling kit (Invitrogen). The two labeled mAbs were tested for internalization. Only 8D8 showed significant internalization (FIGS. 5A-D).

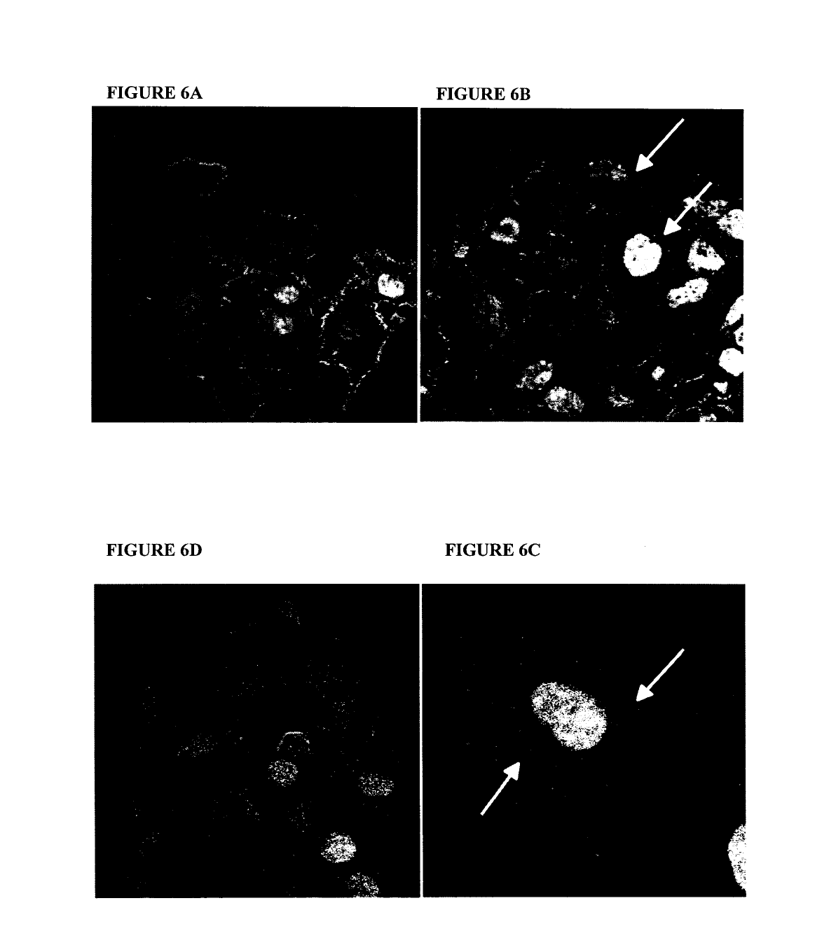

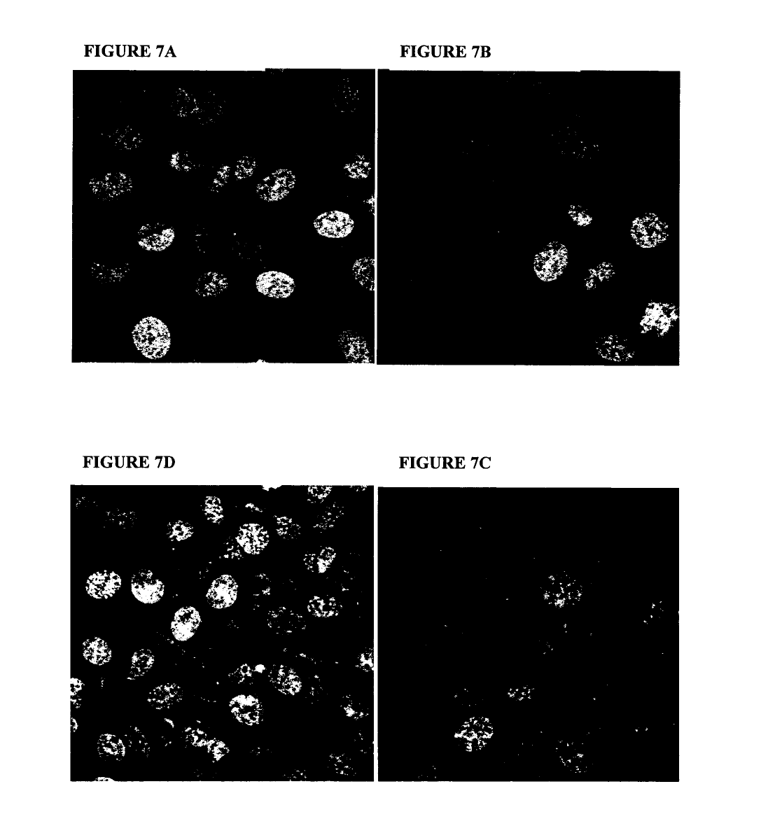

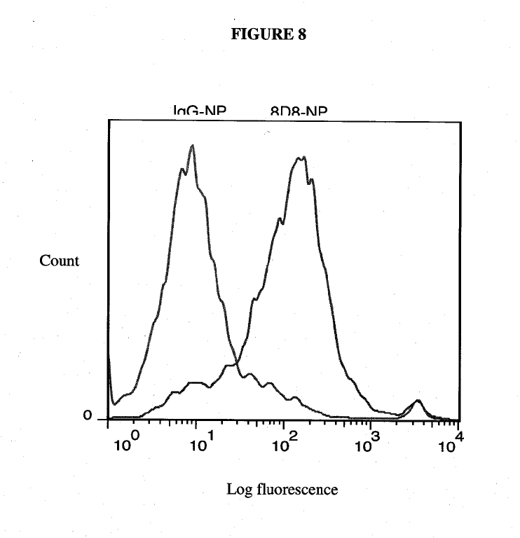

The mAb 8D8 was covalently coated to HA-lipid particles and the particles were incubated with the A549, NRK-naïve and NRK ENDO180 cells to achieve internalization. The 8D8-coated lipid particles incubated at 37° C. with A549 exhibited significant internalization into the cells compared to lipid particles without the coating (FIG. 6) and with 8D8 coated lipid particles, which were incubated with the cells at 4° C. No internalization was observed with the NRK naïve cells (FIG. 7).

8D8-NPs and isotype control particles (IgG-NPs) entrapped with a model small molecule drug (DOX) were prepared as detailed above (composition 1). The 8D8 mAb and separately the isotype control mAb were labeled with Alexa 488 and purified using a desalting column. The mAbs were then conjugated to the NPs via NHS and purified using size exclusion column (see experimental section). Binding to NRK-ENDO180-expressing cells was determined using flow cytometry. As shown in FIG. 8, the binding of 8D8-NPs was high and a clear shift in the fluorescence was observed compare to control particles (IgG-NPs).

### Cell Specific Delivery of DOX Via 8D8-NPs

To examine the selective delivery of a drug (doxorubicin, DOX) using 8D8-NPs, cells expressing ENDO180 (NRK ENDO180+1+) and cells lacking the receptor (NRK ENDO180−/−) were cultured and incubated with a low dose of DOX for 30 min at 37° C. or incubated with the same dose entrapped in 8D8-NPs or IgG-NPs. The cells were washed extensively and incubated with drug-free media to simulate in vivo conditions. Without wishing to be bound to theory, the 8D8-NPs bind tightly to the ENDO180 receptor, are internalized into the cell and do not wash off as do the controls. Cell survival was detected using a cell survival assay (XTT).

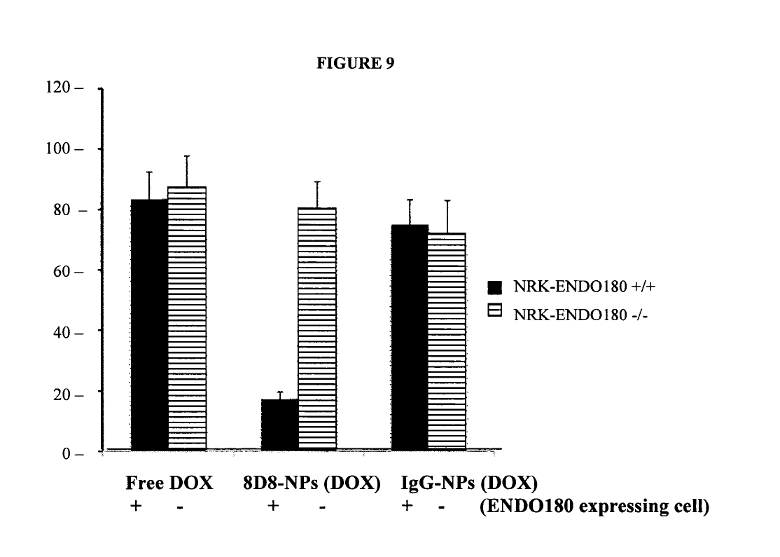

As shown in FIG. 9, the delivery of DOX to ENDO180-expressing cells was selective using the 8D8-NPs. Minimal non-specific uptake was shown when IgG-NPs or when 8D8-NPs were used in NRK cells lacking the ENDO180 receptor.

Binding of 8D8-NPs to NRK ENDO180-Expressing Cells Using NP with HA Spacer.

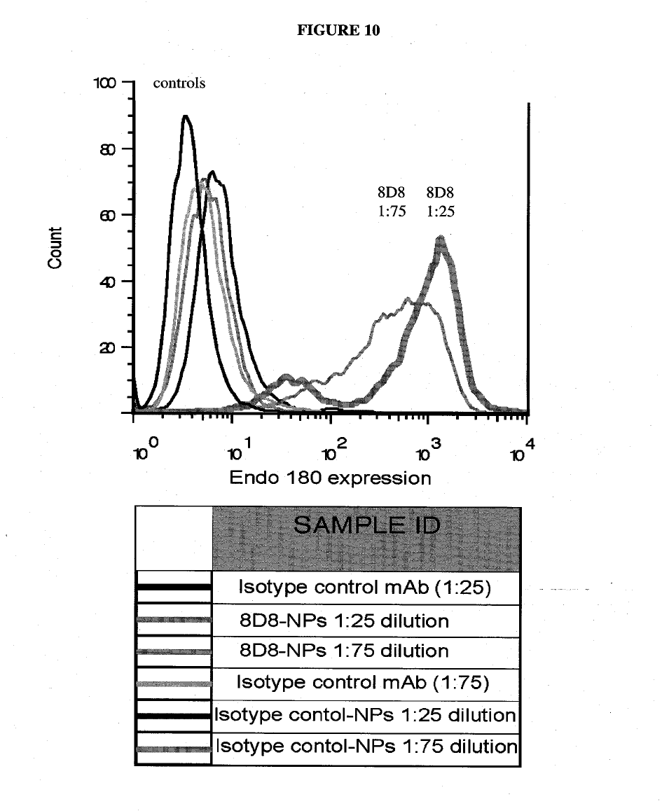

8D8-NPs and isotype control particles (IgG-NPs) entrapped with siRNA were prepared using HA spacer (i.e. composition 1) (see schematic illustration in FIG. 1). Each of the 8D8 mAb and the isotype control mAb were labeled with Alexa 488 and purified using a desalting column. The mAbs were then conjugated to the NPs via EDC and NHS and purified using size exclusion column (see experimental section). Binding to NRK-ENDO180-expressing cells was determined using flow cytometry (See FIG. 10). As shown in FIG. 10, the binding of 8D8-NPs prepared with HA spacer was extremely high and a clear shift in the fluorescence was observed compared to control particles (IgG-NPs).

8D8-NPs (Composition 3) Deliver siRNA to NRK-ENDO180+/+Cells.

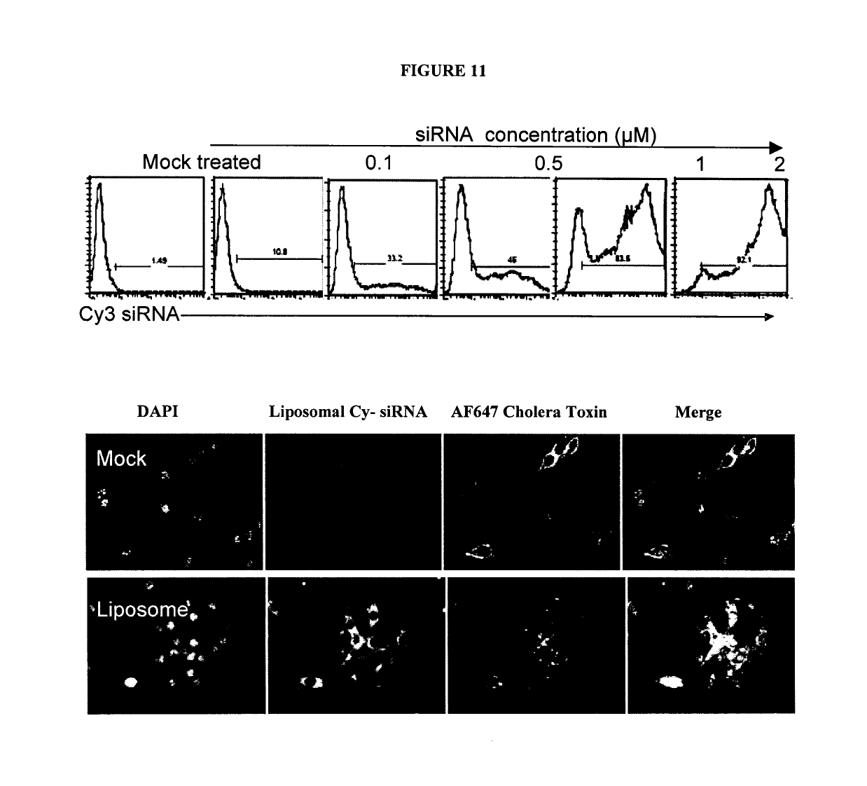

To examine the ability to deliver siRNA into NRK-ENDO180 expressing cells, siRNAs were entrapped in lipid-nanoparticles coated with 8D8 mAb via a HA spacer. Cells were incubated for 1 h with different siRNA concentrations ranging from 0, 0.1, 0.25, 0.5, 1 and 2 μM siRNA. Cells were washed and subjected to flow cytometry (FIG. 11A). In addition in the high siRNA concentration (2 μM), cells were also viewed using fluorescence microscopy (FIG. 11B). A dose response curve of Cy3-siRNA delivery to NRK-ENDO180-expressing cells is shown in FIG. 11A. The delivery was specific with a high content (>90%) of Cy3-siRNA in the higher dose. The results were mirrored by the fluorescence microscopy images demonstrating selective delivery using 8D8-NPs.

8D8-NPs Deliver Cy3-siRNAs into NRK-ENDO180-Expressing Cells and the siRNAs are Localized to the Perinuclear Foci.

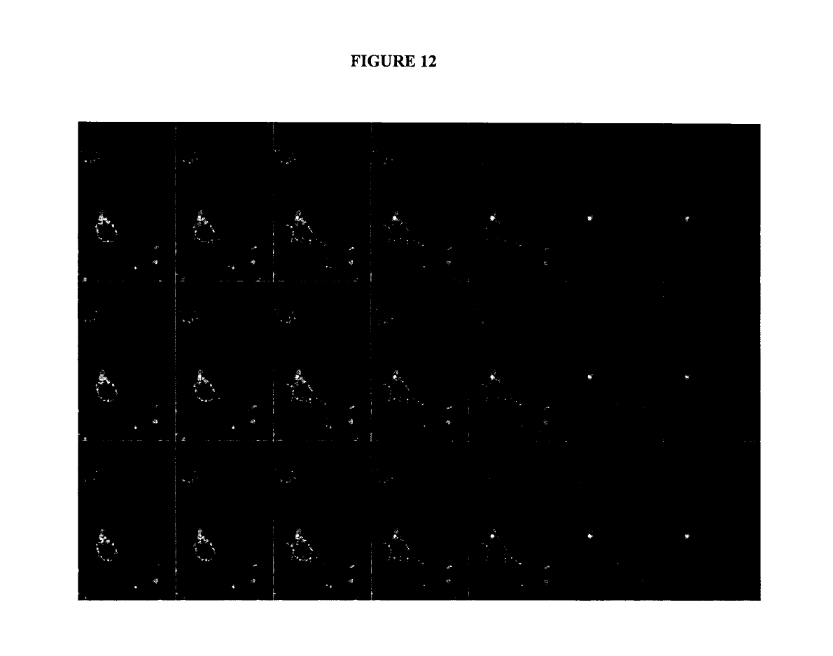

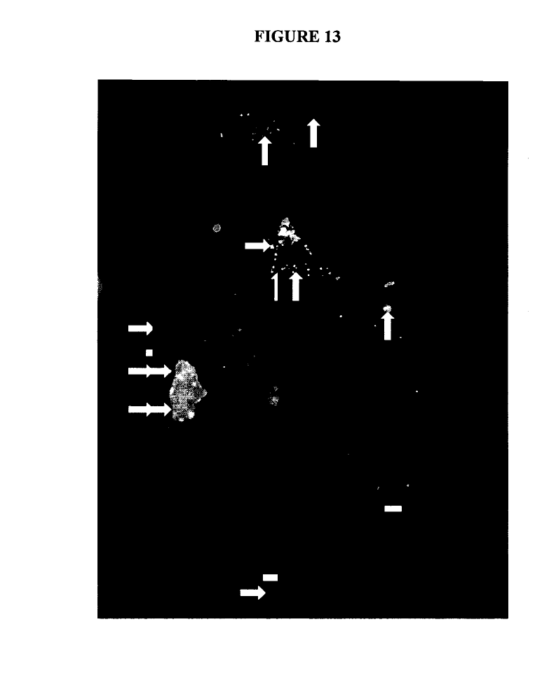

Confocal microscopy analysis (FIGS. 12 and 13) revealed that the Cy3-siRNAs that were delivered via 8D8-NPs are in fact located inside the cells (FIG. 12) and are localized to the perinuclear foci, where the RNAi machinery is also located (FIG. 13—see white arrows pointing the perinuclear foci).

These results demonstrate the ability of 8D8-NPs to selectively deliver cargo (small molecules, as represented by DOX, and dsRNA as represented by Cy3-siRNAs) directly into ENDO180-expressing cells.

### Therapeutic Benefit of 8D8-Coated Particles in A549 Cells.

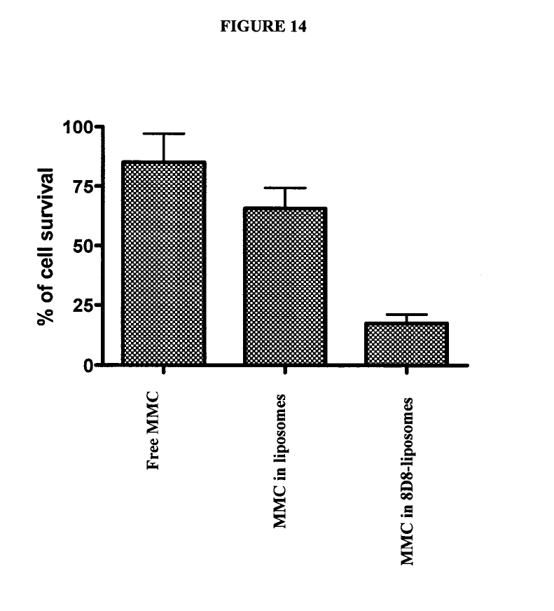

The therapeutic benefit of 8D8-coated particles in A549 cells was compared to non-targeted, regular nano-lipid particles. Mitomycin C (MMC) was used as a therapeutic cargo. MMC was incorporated into the lipid particles in a swelling solution as previously demonstrated (Peer & Margalit, Int J Cancer 108, 780-789 (2004); Bachar, et al. Biomaterials 32, 4840-4848 (2011)) for both liposomes and other lipid-based nanoparticles. 8D8-coated particles (composition 1), regular particles and free MMC all at a concentration of 50 μg/mL were incubated with A549 cells for 1 h at 37° C. After 1 h, cells were washed twice with PBS and incubated for an additional 72 h with drug-free medium. FIG. 14 shows the therapeutic benefit of using a targeted version vs. free drug, or uncoated nanoliposomes. Without wishing to be bound by theory, the therapeutic benefit is due to the specific uptake of the 8D8-coated lipid particles by the cells and release of their MMC payloads in target cells. In contrast to the effect of small, non-coated liposomes that do not internalize well into these cells and thus are washed away after 1 h incubation. The binding of the 8D8 coated nanoparticles to the ENDO180 receptor and the active recycling process is speculated to be the major denominator of results observed in these cells.

### Example 4

### In Vitro Knockdown of Target Gene with 8D8 Coated Particles Carrying siRAC1

The A549 cell line was used as the cancer cell model. Cells were seeded into six wells cell culture plates at 7.0×10⁵ cells/well in RPMI medium, supplemented with antibiotics, L-Glutamine and 10% fetal calf serum (Biological Industries, Beit Haemek, Israel). 24 hours post seeding the medium was removed and replaced with RPMI medium with glutamine and 10% serum, without antibiotics. The cells were transfected with 8d8-HA-NPs or with IgGCtrl-HA-NPs encapsulating CY5-labeled Rac1_{—}28 or eGFP siRNAs. As a positive control, Oligofectamine (Invitrogen) was used according to the manufacturer's instructions. One hour post incubation medium was removed and cells were washed and supplemented with complete medium. Six days after transfection the cells were split 1:3. The final siRNA concentrations applied to the cells in the lipid-nanoparticles was 20-100 nM. Six days after transfection, total RNA was isolated using the EzRNA RNA purification kit (Biological industries, Beit Haemek, Israel). 1 μg of RNA was reverse transcribed into cDNA using the High Capacity cDNA Reverse Transcription Kit (Applied Biosystems, Foster City, Calif.), Quantification of cDNA (5 ng total) was performed on the step one Sequence Detection System (Applied Biosystems, Foster City, Calif.), using Syber green (Applied Biosystems). GAPDH was chosen as a housekeeping gene.

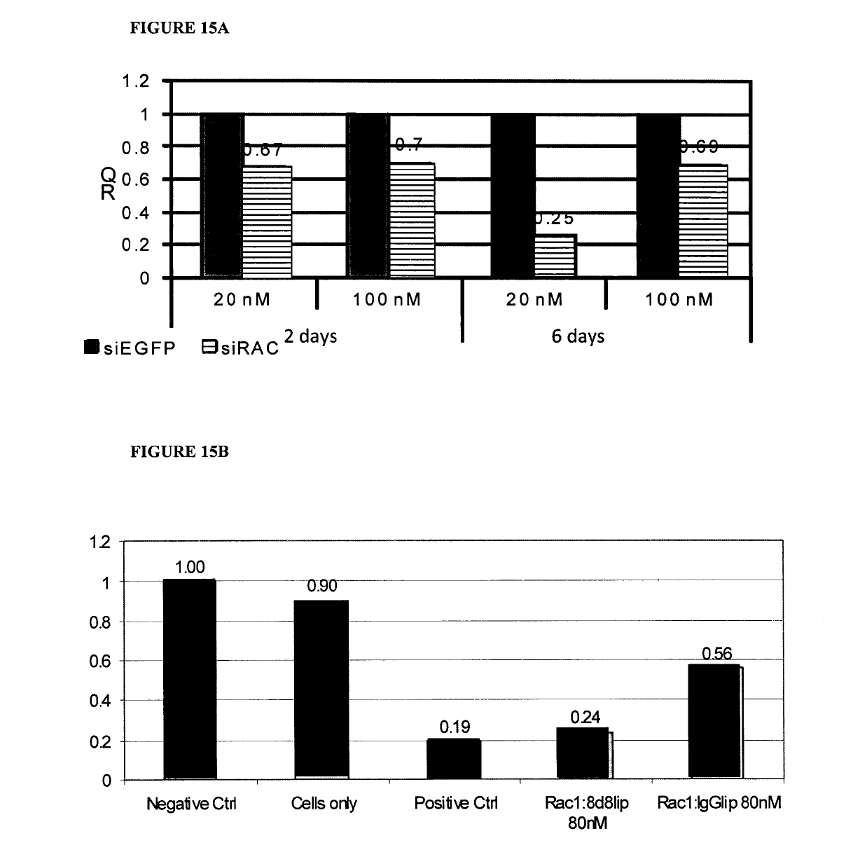

In vitro results are shown in FIGS. 15A-15B. FIGS. 15A and 15B show in vitro knock down of Rac1 mRNA (levels of residual mRNA shown) in a A549 cell line exposed to 8D8-NPs encapsulating siRNA to RAC. FIG. 15A shows knock down after 2 and 6 days. FIG. 15B shows knock down after 6 days. Rac1:8d8lip refers to 8D8 coated lipid nanoparticles encapsulating siRAC1. Rac1:IgGlip refers to IgG coated lipid nanoparticles encapsulating siRAC1.

EGFP (enhanced Green Fluorescent Protein) siRNA has the following structure: a sense strand GCCACAACGUCUAUAUCAU (SEQ ID NO:9) with unmodified ribonucleotides in positions 1, 3, 5, 7, 9, 11, 13, 15, 17 and 19 and 2′O-Methyl sugar modified ribonucleotides in positions 2, 4, 6, 8, 10, 12, 14, 16 and 18; and antisense strand 5′ AUGAUAUAGACGUUGUGGC 3′ (SEQ ID NO:10) with unmodified ribonucleotides in positions 2, 4, 6, 8, 10, 12, 14, 16 and 18 and 2′O-Methyl sugar modified ribonucleotides in positions 1, 3, 5, 7, 9, 11, 13, 15, 17 and 19, and a Cy5 fluorescent moiety covalently attached to the 3′ terminus.

siRNA identified as RAC1_{—}28_S1842 (BioSpring, Frankfurt, Del.) target the RAC1 gene and has the following strands: Sense strand 5′ GUGCAAAGUGGUAUCCUA 3′ (SEQ ID NO:11), with unmodified ribonucleotides in positions 2, 4, 6, 7, 8, 9, 11, 12, 14, 15, 17 and 19 and 2′O Methyl sugar modified ribonucleotides in positions 1, 3, 5, 10, 13, 16 and 18. Antisense strand: 5′ UAGGAUACCACUUUGCACG 3′ (SEQ ID NO:12) with unmodified ribonucleotides in positions 2, 3, 4, 5, 7, 8, 10, 12, 14, 16 and 18 and 2′O Methyl sugar modified ribonucleotides in positions 1, 6, 9, 11, 13, 15, 17 and 19, and a Cy5 fluorescent moiety covalently attached to the 3′ terminus.

### Example 5

### Biodistribution of ENDO180 Targeting Nanoparticles in Tumor Bearing Athymic Nude Mice

Objective: To assess formulated Cy5-labeled RAC1_{—}28_S1842 siRNA biodistribution (BD) in A549 (adenocarcinoma human alveolar basal epithelial cells) tumor bearing athymic nude mice (TBM).

### Material and Methods:

Test article: siRNA identified as RAC1_{—}28_S1842 (BioSpring, Frankfurt, Del.). 30.179 mg siRNA were dissolved in 1.50 lml water for injection (WFI, Norbrook) to achieve a stock solution of 20 mg/ml. 0.35 ml of the stock solution was lyophilized to 7 mg which were dissolved in 14 ml DEPC-treated water to achieve a stock solution of 0.5 mg/ml.

Formulated RAC1_{—}28_S1842 in uncoated NPs: The uncoated NPs were composed of Pure Soybean phosphatidylcholine (Phospholipon 90G, Phospholipid GMBH Germany). 1,2-dipalmitoyl-sn-glycero-3-phosphoethanolamine (DPPE) and Cholesterol (Chol) (Avanti Polar Lipids Inc. (Alabaster, Ala., USA)). PC:Chol:DPPE at a molar ratio of about 60:20:19.9. The lipids were dissolved in ethanol, evaporated until dry under reduced pressure in a rotary evaporator (Buchi Rotary Evaporator Vacuum System Flawil, Switzerland). Following evaporation, the dry lipid film was hydrated in 10 ml of HEPES (pH 7.4), followed by extensive agitation using a vortex device and 2 hr incubation in a shaker bath at 65° C. The MLV were extruded through a Lipex extrusion device (Northern Lipids, Vancouver, Calif.), operated at 65° C. and under nitrogen pressures of 200-500 psi. Extrusion was carried out in stages using progressively smaller pore-size polycarbonate membranes (Whatman Inc, UK), with several cycles per pore-size, to achieve unilamellar vesicles (ULV) in a final size range of ˜100 nm in diameter. The obtained NPs were lyophilized until completely dry (48 hours). The lyophilized particles were hydrated with DEPC-treated water with 0.5 mg/ml siRNA RAC1_{—}28_S1842.

Formulated RAC1_{—}28_S1842 in HA coated NPs: High molecular weight Hyaluronan (HA), 700 KDa (Lifecore Biomedical LLC Chaska, Minn., U.S.A) was dissolved in 0.2M MES buffer (pH 5.5) to a final concentration of 5 mg/ml. HA was activated with EDC and sulfo-NHS at a molar ratio of 1:1:6. After 30 minutes of activation the unilamellar vesicles were added and the pH was adjusted to 7.4. The solution was incubated at room temperature (2 hr). The free HA was removed by 3 cycles of repeated washing by centrifugation (1.3×105 g, 4° C., 60 min). The obtained HA coated NPs were lyophilized until completely dry (48 hours). The lyophilized particles were hydrated with DEPC-treated water with 0.5 mg/ml siRNA RAC1_{—}28_S1842.

Formulated RAC1_{—}28_S1842 in anti-ENDO180-HA coated NPs: ENDO180 8D8 antibody and mouse IgG control (I 8765), were concentrated to a final concentration of 10 mg/ml (Centricon Centrifugal Filter units). 20 μl was activated with 1.2 μg of EDC and 1.44 μg of sulfo-NHS (pH 5.5). After incubation at RT for 30 minutes, 0.8 mg of HA coated NPs (See above, the HA coated NPs added before their lyophilization) were added to the activated selected antibodies (Ab) and the pH adjusted to pH7.4. Liposomes were incubated ON at 4° C. Liposomes and free antibodies were separated on CL-4B column. The solution was incubated at room temperature (2 hr). The free HA was removed by 3 cycles of repeated washing by centrifugation (1.3×10⁵ g, 4° C., 60 min). The obtained 8D8-HA coated NPs were lyophilized until complete water removal was ensured (48 hours). The lyophilized particles were hydrated with DEPC-treated water with 0.5 mg/ml siRNA RAC1_{—}28_S1842.

HBSS refers to vehicle: 150 mM NaCl, 20 mM Hepes, pH=7.4

Test system: Species/Strain: athymic nude mice (Harlan); 11 weeks old females; Body Weight Range: 20-22 gr., Group Size: 1-3; Total number of animals in the study: 36 out of 40 tumor injected mice

Animal Husbandry: Animals were provided an ad libitum commercial rodent diet regular chow, and free access to drinking water.

Environment: (i) Acclimatization of at least 5 days.

(ii) All the animals were confined in a limited access facility with environmentally-controlled housing conditions throughout the entire study period, and maintained in accordance with approved standard operating procedures (SOPs).

Cells: A549 (Adenocarcinomic Human Alveolar Basal Epithelial Cells) (ATCC# CCL-185)

One week after arrival. 40 athymic nude mice were injected subcutaneously with A549 cells into the flank region. The mice were checked visually for tumor progression and discomfort on a daily basis. Upon reaching sufficient tumor volume of approximately 5 mm the mice were injected i.v. with different formulated RAC1_{—}28_S1842 siRNA (un coated, HA-coated and 8D8-HA) according to the study design, in Table 2, hereinbelow.

TABLE 2

| | | Treatment | | | | | |
|---|---|---|---|---|---|---|---|
| Group | | | | Dosage | Admin | Termination | Group |
| no. | Group | siRNA | Formulation | (μg) | route | (hours) | Size |
| 1 | Control | none | HBSS | 200 μl | I.V. | 6, 24 | 3, 3 |
| | (HBSS | | | | | | |
| | injected) | | | | | | |
| 2 | Uncoated | RAC1_28_S1842 | PC:Chol:DPPE | 100 μg/ | I.V. | 6, 24 | 3, 3 |
| | NPs-RAC1 | | | 200 μl | | | |
| 3 | HA-NPs- | RAC1_28_S1842 | Hyaluronan- | 100 μg/ | I.V. | 6, 24 | 3, 3 |
| | RAC1 | | Coated- | 200 μl | | | |
| | | | PC:Chol:DPPE | | | | |
| 4 | 8d8-HA- | RAC1_28_S1842 | ENDO180- | 100 μg/ | I.V. | 6, 24 | 3, 3 |
| | NPs-RAC1 | | 8D8- | 200 μl | | | |
| | | | Hyaluronan- | | | | |
| | | | PC:Chol:DPPE | | | | |
| 5 | Naked | RAC1_28_S1842 | Water | 100 μg/ | I.V. | 6, 24 | 3, 3 |
| | RAC1 | | | 200 μl | | | |

Preparation of Tumor cell suspensions: 0.5×10⁶ A549 cells (adenocarcinomic human alveolar basal epithelial cells) per mouse.

Tumor induction: The cell suspension, at a concentration of 2.5×10° cells/ml, was injected by a single administration subcutaneously (sc) into the flank region of each animal, using a 27G needle. Administration was performed as soon as possible following cell preparation.

Test Article Preparation: On the day of the experiment all carrier formulations (un coated, HA-coated and 8D8-HA) were lyophilized and stored in glass bottles in batches (−20° C.). Prior to the experiment, a single dose of lyophilized particles was taken, rehydrated and checked for size by dynamic light scattering. The lyophilized carriers were rehydrated with siRNA (0.5 mg/ml) dissolved in DEPC-treated water, siRNA to lipid ratio 1:2. After 30 minutes of mild shaking on an orbital shaker at room temperature, the carriers were injected i.v. into the mice.

Test Article Administration: The single intravenous (iv) administration was performed at 30 days post tumor inoculation. Formulated siRNA in a dose of 0.5 mg/1 ml, injection volume 200 μL using a 27G injection needle.

After the cell injection and the carrier injection, the mice were checked daily for signs of distress and tumor growth. Post mortem examination was performed with the Maestro imaging system of the mice sacrificed after 6 hr the mice that are sacrificed 24 after carrier injection were dissected for biodistribution analysis.

Study termination: 6 hr after test article injection half of the mice were sacrificed with CO₂ (according to rules and regulations of the University) and imaged. At about 24 hr post injection the remaining animals were bled and then sacrificed with CO₂. Organs (tumor, lungs, liver, spleen and kidneys) were removed. One kidney, one liver lobe, half a lung, half a spleen and tumors were flash frozen in liquid nitrogen. The remaining organs were preserved in 4% formaldehyde (1 ml per organ).

### Evaluation and Results

siRNA quantification in tissues and tumor: RAC1_{—}28_S1842 siRNA quantity was examined by stem and loop qPCR. siRNA was detected in the tissue lysates by lysing the samples in 0.25% triton followed by qPCR according to standard methods using SYBR Green method in the Applied Biosystem 7300 PCR System.

RAC1 mRNA levels and RACE analysis in the RNA prepared from all frozen tissues and cells was measured using qPCR. cDNA was prepared according to standard methods. For RACE analysis of the RAC1 cleavage product—RNA will be prepared by total RNA isolation

siRNA distribution was also assessed by in-situ hybridization (ISH).

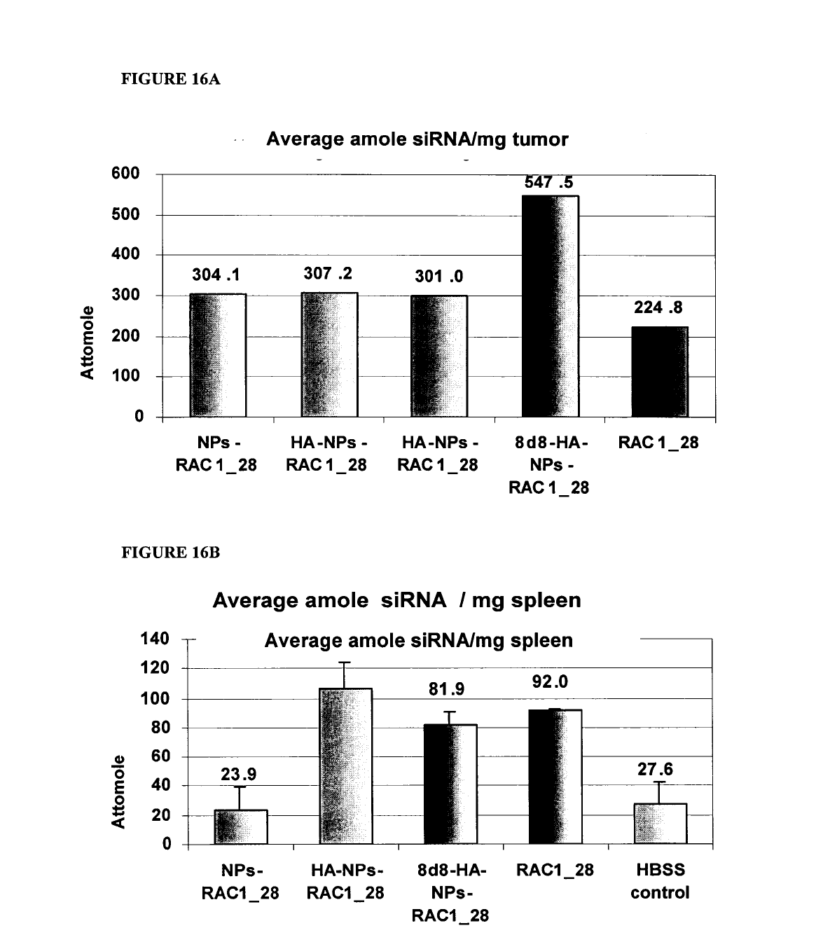

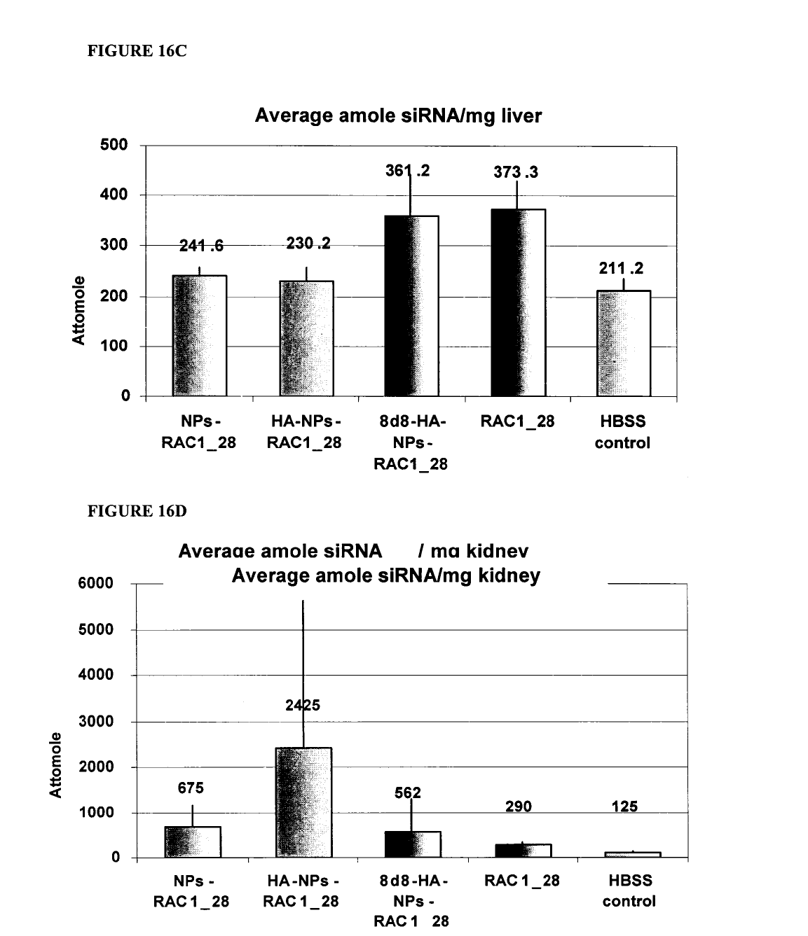

Cy5 labeled siRNA was observed in the tumor, liver and kidneys of tumor bearing mice. Strong Cy5 fluorescence was observed in the tumor and in both kidneys, not shown. High levels of siRNA were observed in the tumor of animals injected with lipid nanoparticles conjugated to the anti-ENDO180 antibody (8D8) via a hyaluronic acid (HA) moiety as shown in the graphs in FIGS. 16A-16D. FIGS. 16A-16D present graphs depicting biodistribution of siRNA to various body organs in mice treated with ENDO180 coated nanoparticles (NPs) encapsulating Cy5-Rac1_{—}28 in a murine cancer model. The amount of siRNA (atomoles) present per mg tissue sample is presented in animals treated with different compositions as follows: nanoparticles encapsulating siRAC1 (NPs-RAC1_{—}28); hyaluronic acid coated nanoparticles encapsulating siRAC1 (HA-NPs-RAC1_{—}28); 8D8 and hyaluronic acid coated nanoparticles encapsulating siRAC1 (8d8-HA-NPs-RAC1_{—}28); siRAC I alone (RAC1_{—}28) in tumors (16A), spleen (16B), liver (16C) and kidney (16D). Spleen, liver and kidney are average from at least 3 mice).

### Example 6

### Biodistribution of ENDO180 Targeting Nanoparticles in Tumor Bearing Athymic Nude Mice

Objective: To assess formulated RAC1_{—}28_S1908 siRNA biodistribution (BD) in A549 (adenocarcinomic human alveolar basal epithelial cells) tumor bearing athymic nude mice (TBM).

Materials and methods:

Test article: siRNA identified as RAC1_{—}28_S1908 (BioSpring, Frankfurt, Del.) target the RAC1 gene and has the following strands:

Sense strand 5′ GUGCAAAGUGGUAUCCUA 3′ (SEQ ID NO:9), with unmodified ribonucleotides in positions 2, 4, 6, 7, 8, 9, 11, 12, 14, 15, 17 and 19 and 2′O Methyl sugar modified ribonucleotides in positions 1, 3, 5, 10, 13, 16 and 18.

Antisense strand 5′ UAGGAUACCACUUUGCACG 3′ (SEQ ID NO:10) with unmodified ribonucleotides in positions 2, 3, 4, 5, 7, 8, 10, 12, 14, 16 and 18 and 2′O Methyl sugar modified ribonucleotides in positions 1, 6, 9, 11, 13, 15, 17 and 19.

Preparation of siRNA: 5 mg dissolved in 5130 DEPC-treated water to obtain a stock solution of 9.75 mg/ml.

Formulated compound 0.4 mg/ml RAC1_{—}28_S1908 in 8d8-HA-NPs (ENDO180-8D8-Hyaluronan-PC:Chol:DPPE): ENDO180 mAb 8D8, was concentrated to a final concentration of 10 mg/ml (Centricon Centrifugal Filter units). 20 μl were activated with 1.2 μs of EDC and 1.44 μg of sulfo-NHS (pH 5.5). After incubation at RT for 30 min, 0.8 mg of HA coated NPs. The uncoated NPs were PC:Chol:DPPE at molar ratios of about 60:20:19.9. The lipids were dissolved in ethanol, evaporated to dryness under reduced pressure in a rotary evaporator. Following evaporation, the dry lipid film was hydrated in 10 ml of HEPES (pH 7.4) followed by extensive agitation (vortex) and 2 hr incubation in a shaker bath at 65° C. The MLV were extruded through a Lipex extrusion device operated at 65° C. and under nitrogen pressures of 200-500 psi. The extrusion was carried out in stages using progressively smaller pore-size polycarbonate membranes (Whatman Inc, UK), with several cycles per pore-size, to obtain ULV at a final size range of −100 nm in diameter. The liposomes were added to the activated selected antibodies (Ab) and the pH adjusted to pH 7.4. Liposomes were incubated overnight (O.N) at 4° C. Liposomes and free antibodies were separated on CL-4B column. The solution was incubated 2 hr at room temperature. Free HA was removed by 3 cycles of repeated washing by centrifugation (1.3×10⁵ g, 4° C., 60 min). The 8D8-HA coated NPs were lyophilized until completely dry (48 hr). A portion of 1 mg lyophilized particles were hydrated with 20.5 μl stock RAC1_{—}28_S1908_S18 siRNA of 9.75 mg/ml (200 μg) and 479.5 μl DEPC-treated water to obtain 500 μl of 0.4 mg/ml siRNA in 8d8-HA-NPs. This prepared siRNA stock was used in 2 mice. This procedure was repeated 3 times.

Formulated compound, control antibody coated NPs: 0.4 mg/ml RAC1_{—}28_S1908 in NMIgG-HA-NPs (NMIgG Hyaluronan-PC:Chol:DPPE): Description of the test material: mouse IgG control (I 8765), was concentrated to a final concentration of 10 mg/ml (Centricon Centrifugal Filter units). 20 μl was activated with 1.2 μg of EDC and 1.44 μg of sulfo-NHS (pH 5.5). After incubation at RT for 30 minutes, 0.8 mg of HA coated NPs were added to the activated selected antibodies (Ab) and the pH adjusted to pH7.4. Liposomes were incubated overnight (O.N) at 4° C. Liposomes and free antibodies were separated on CL-4B column. The solution was incubated at room temperature (2 hr). The free HA was removed by 3 cycles of repeated washing by centrifugation (1.3×105 g, 4° C., 60 min). The obtained IgG-HA coated NPs were lyophilized until complete water removal was ensured (48 hours). A portion of 1 mg lyophilized particles were hydrated with 20.5 μl stock RAC1_{—}28_S1908_S18 siRNA of 9.75 mg/ml (200 μg) and 479.5 μl DEPC-treated water, to obtain. To 500 μl of 0.4 mg/ml siRNA in NMIgG-HA-NPs. This prepared siRNA stock was administered to 2 mice. This procedure was repeated 3 times.

HBSS refers to vehicle: 150 mM NaCl, 20 mM Hepes, pH=7.4

Test system: Species/Strain: athymic nude mice (Harlan); 11 weeks old females; Body Weight Range: 20-22 gr., Group Size: 5-8; Total number of animals in the study 18.

Animal Husbandry and cell line: as provided in Example 5, supra.

Two weeks after acclimatization, the athymic nude mice were injected subcutaneously into the flank region with A549 cells. The mice were checked visually for tumor progression and discomfort on a daily bases. Upon reaching sufficient tumor volume of approximately 5 mm the mice are injected i.v. with 4 mg/kg of different formulated RAC1_{—}28_S1908 siRNA (8D8-HA and IgG Ctrl complex formulations) according to the study design Table 3 (T=0).

At about 24 hr post siRNA/carrier injection (T=24 h) another dose of 4 mg/kg siRNA/carrier was injected. At about 48 hr post 1^{st} siRNA/carrier injection (T=48 h) animals were bled and then sacrificed with CO₂. Organs (tumor, lungs, liver, spleen and kidneys) were collected.

TABLE 3

| | | Treatment | | | | | | |
|---|---|---|---|---|---|---|---|---|
| | | | | | | | Term. | |
| | | | | | | | after 2^{nd} | |
| Group | Group | | | Dosage | Admin | Injection | injection | Group |
| No. | Title | siRNA | Formulation | (μg) | route | days | (hours) | Size |
| 1 | 8d8-HA- | RAC1_28_ | ENDO180-8D8- | 2 × 80μg/ | I.V. | 0, 1 | 48 | 8 |
| | NPs-RAC1 | S1908 | Hyaluronan- | 200 μl | | | | |
| | | | PC:Chol:DPPE | | | | | |
| 2 | HA- | RAC1_28_ | NMIgG- | 2 × 80 μg/ | I.V. | 0, 1 | 48 | 7 |
| | IgGCtrl- | S1908 | Hyaluronan- | 200 μl | | | | |
| | NPs-RAC1 | | PC:Chol:DPPE | | | | | |
| 3 | Control | N/A | HBSS | 2 × 200 μl | I.V. | 0, 1 | 48 | 5 |
| | HBSS | | | | | | | |

Preparation of Tumor Cells: Tumor cells suspensions: 2.0×10⁶ A549 (adenocarcinomic human alveolar basal epithelial cells) per mouse.

Tumor induction: The cell suspension, at concentration of ˜10⁶ cells/ml, was injected subcutaneously (sc) into the flank region of each animal at dose volume of 0.2 ml/animal using a 27G needle. Administration was performed as soon as possible following cell preparation.

Monitoring After injection, the mice were checked visually for tumor progression and discomfort on a daily basis. Tumor size was monitored measured and recorded. When tumor volume reached approximately 5 mm the mice were sorted into 3 groups.

Test Article Preparation: Prior to the experiment, all carrier formulations (IgGCtrl-HA-coated and 8D8-HA-coated) were lyophilized and stored in glass bottles in batches (−20° C.). a single dose of lyophilized particles was taken, rehydrated and checked for size by dynamic light scattering. On the day of the experiment 1 mg of lyophilized carriers (0.5 mg per mouse per single dose) were rehydrated with siRNA and DEPC-treated water, siRNA to lipid ratio 1:10. After 30 minutes of mild shaking in an orbital shaker at room temp to insure complete dissolvent, the carriers were injected i.v. into the mice (200 μl, 4 mpk).

Test Article Administration: The single intravenous (i.v.) administration is performed at 14 days post tumor inoculation. Formulated siRNA at a dose of 0.32 mg/ml, injection volume 2504 using a 27G injection needle. A second i.v. administration was performed 24 h after the first iv injection in the same manner.

Study termination 48 hr after the first test article/vehicle injection all mice were bled and then sacrificed with CO₂. Organs (tumor, lungs, liver, spleen and kidneys) were collected.

Plasma separation Blood samples were centrifuged for 15 min at 1000 g at RT. Plasma was immediately frozen in liquid nitrogen. All plasma samples will be kept in −80° C. until qPCR.

Tissue collection for qPCR and ISH: Frozen tissues were collected from 6 mice of both group 2 and 3 and 4 mice from group 1. Fixed tissues were collected from 2 mice from group 1 and one mouse both group 2 and 3.

For Frozen Tissues: Both kidneys, lungs, liver, spleen and tumor were harvested, collected into pre labeled tubes and immediately snap frozen in liquid nitrogen.

Tumor Collection for Histopathology (Groups 1-3). Tumors from two animals of groups 1, one animal of group 2 and one animal of group 3 were collected and immediately placed in 10% formalin (each tumor separately in 15 ml formalin tube) pH 7.4 and paraffin embedded for slide preparation. Other organs of these animals were collected and snap frozen in liquid nitrogen.

### Evaluation and Results

siRNA quantification in tissue and tumor: RAC1_{—}28_S1908 siRNA quantity was examined by stem and loop qPCR. siRNA was detected in the tissue lysates by lysing the samples in 0.25% triton followed by qPCR according to standard methods using SYBR Green method in the Applied Biosystem 7300 PCR System.

RAC1 mRNA levels and RACE analysis in the RNA prepared from all frozen tissues and cells were measured using qPCR. cDNA was prepared according to standard and qPCR was performed as described above. For RACE analysis of the RAC1 cleavage product—RNA was prepared by total RNA isolation using EZ RNA kit.

In situ hybridization siRNA distribution will be performed to detect RAC1_{—}28 siRNA in the various tissue samples.

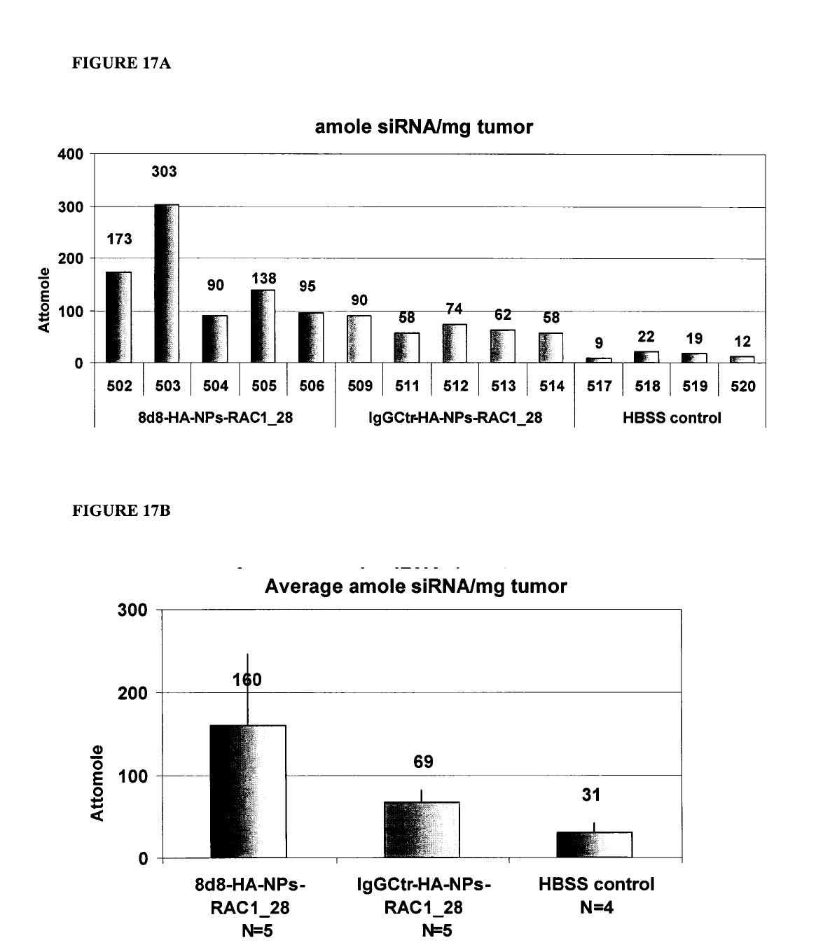

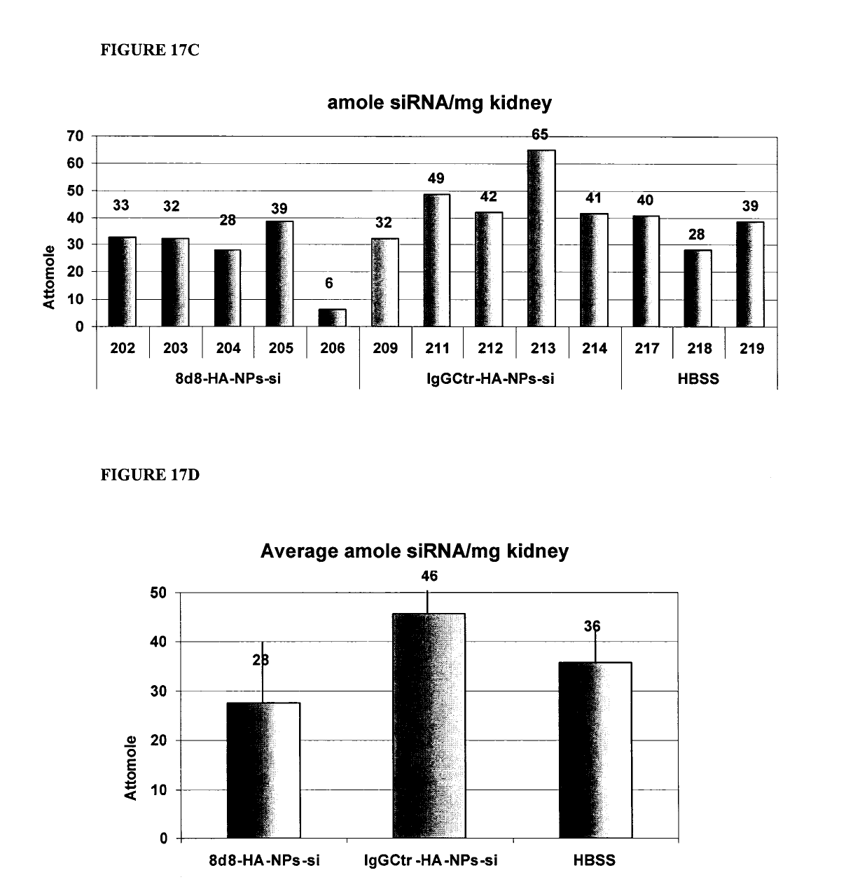

siRNA was observed in the tumor, liver and kidneys of tumor bearing mice. High levels of siRNA were observed in the tumor of animals injected with lipid nanoparticles conjugated to the anti-ENDO180 antibody (8D8) via a hyaluronic acid (HA) moiety as shown in the graphs in FIGS. 17A-17D. FIGS. 17A-17D present graphs depicting biodistribution of ENDO180 coated nanoparticles (NPs) encapsulating Rac1_{—}28 in the tumor and kidneys from a murine cancer model. The amount of siRNA (atomoles) present per mg tissue sample is presented in animals treated with different compositions as follows: 8D8 and hyaluronic acid coated nanoparticles encapsulating siRAC1 (8d8-HA-NPs-si); IgG and hyaluronic acid coated nanoparticles encapsulating siRAC1 (IgGCtr-HA-NPs-si); siRAC1_{—}28 in buffer (HBSS) in tumors (17A and 17B) and kidneys (17C and 17D). “n” refers to number of animals included in average (17B and 17D).

### Example 7

### siRNA Activity

Efficacy of lipid nanoparticles encapsulating siRNA to knock down target gene or cleave target mRNA is assessed using standard methods known by persons with skill in the art and include measurements of residual mRNA levels and residual protein levels and RACE (cleavage).

Although the examples utilize a limited number of siRNA molecules, it is to be understood that the compositions as disclosed herein are formulated to encompass oligonucleotides including antisense molecules, dsRNA, siRNA and the like that target any gene in an organism (i.e. inhibits gene expression/down-regulates gene expression) and preferably genes associated with disease, where inhibition/down-regulation of such a gene would be beneficial to the organism.

The methods and compositions disclosed herein have been described broadly and generically. Each of the narrower species and subgeneric groupings falling within the generic disclosure also form part of the disclosure. This includes the generic description of the disclosure with a proviso or negative limitation removing any subject matter from the genus, regardless of whether or not the removed material is specifically recited herein. Other embodiments are within the following claims.

1. A composition comprising a) a lipid-based carrier moiety; b) an ENDO180 targeting moiety; and c) an effective amount of a therapeutic agent or a diagnostic agent or a combination thereof; wherein the carrier moiety and the targeting moiety are covalently bound.

2. The composition of claim 1, wherein the carrier moiety and the targeting moiety are covalently bound via a surface modification of the carrier moiety with a synthetic polymer, a natural polymer or a semi synthetic polymer.

3. The composition of claim 2, wherein the synthetic polymer comprises a PEG moiety.

4. The composition of claim 3, wherein the PEG moiety comprises NHS-PEG-DSPE [3-(N-succinimidyloxyglutaryl)aminopropyl, polyethyleneglycol-carbamyl distearoylphosphatidyl-ethanolamine].

5. The composition of claim 2, wherein the natural polymer comprises a polysaccharide or a glycosaminoglycan.

6. The composition of claim 5, wherein the glycosaminoglycan comprises hyaluronic acid.

7. The composition of claim 1, wherein the ENDO180 targeting moiety comprises an ENDO180 binding protein that binds an extracellular domain of an ENDO180 polypeptide present on a cell and is internalized into the cell by the ENDO180 polypeptide.

8. The composition of claim 7, wherein the ENDO180 binding protein comprises an ENDO180 antibody or a functional fragment thereof capable of binding ENDO180.

9. (canceled)

10. The composition of claim 8, wherein the ENDO180 antibody or a functional fragment thereof is selected from the group consisting of:
a. an isolated monoclonal antibody or an antigen-binding fragment thereof, produced by the hybridoma cell line E3-8D8 deposited with the BCCM under Accession Number LMBP 7203CB;
b. an antibody or an antigen-binding fragment thereof that binds to the same epitope as the antibody of (a);
c. a humanized version of the antibody or an antigen-binding fragment thereof of (a), or a humanized version of the antibody or antigen-binding fragment of (b);
d. a chimeric version of the antibody or an antigen-binding fragment thereof of (a), or a chimeric version of the antibody or antigen-binding fragment of (b);
e. a recombinant polypeptide or antigen-binding fragment thereof comprising the antigen binding domain of the antibody of (a) which is internalized in to a cell by the ENDO180 receptor;
f. an antigen-binding fragment of an antibody comprising a polypeptide substantially similar to SEQ ID NO: 6; and
g. a recombinant polypeptide comprising CDRs having an amino acid sequence substantially similar to amino acid sequences set forth in SEQ ID NO:7 and 8.

11. (canceled)

12. The composition of claim 1, wherein the lipid-based carrier moiety comprises a lipid particle.

13. The composition of claim 12, wherein the lipid particle comprises one or more lipids selected from the group consisting of phosphatidylcholine or a derivative thereof, phosphatidylglycerol or derivative thereof, and phosphatidylethanolamine (PE) or a derivative thereof; or a combination thereof.

14. The composition of claim 12, wherein the lipid particle further comprises one or more cationic lipid and/or cholesterol.

15. (canceled)

16. The composition of claim 12, wherein the lipid particle comprises dioleoyl phosphatidylethanolamine (DOPE) and cholesterol, possibly further comprising hydrogenated soy phosphatidylcholine (HSPC).

17. (canceled)

18. The composition of claim 13, wherein the lipid particle comprises DOPE, Hydrogenated soybean phosphatidylcholine (HSPC), cholesterol (Chol) and the PEG moiety NHS-PEG-DSPE at a molar ratio of about 4.5:20:75:0.5 (DOPE:HSPC:Chol:NHS-PEG-DSPE); or wherein the lipid particle comprises Dioleoyl Phosphatidylethanolamine (DOPE), 1,2-di-O-octadecenyl-3-trimethylammonium propane (DOTMA) and cholesterol (Chol) at a molar ratio of about 4:2:1 (DOPE:DOTMA:Chol).

19. (canceled)

20. (canceled)

21. The composition of claim 14, wherein the lipid particle comprises DPPE and cholesterol, possibly further comprising soy PC.

22. (canceled)

23. The composition of claim 21, wherein the lipid particle comprises soy PC, DPPE and cholesterol at a molar ratio of about 3:1:1 (soy PC:DPPE:cholesterol).

24. The composition of claim 1, wherein the lipid particle is about 85 to about 200 nM in diameter, preferably about 85 to about 130 nm and/or wherein the lipid particle comprises a zeta potential of about (−7) to about (−40).

25. (canceled)

26. (canceled)

27. The composition of claim 1, wherein the composition comprises at least one therapeutic agent selected from the group consisting of a nucleic acid compound and a non-nucleic acid compound, or a combination thereof.

28. (canceled)

29. (canceled)

30. The composition of claim 27, wherein the nucleic acid is selected from the group consisting of an antisense compound, a chemically modified double stranded RNA compound, an unmodified double stranded RNA compound, a chemically modified shRNA compound, an unmodified shRNA compound, a chemically modified miRNA compound, and an unmodified miRNA compound, a chemically modified siRNA, a chemically unmodified siRNA, and ribozyme, or a combination thereof.

31. (canceled)

32. A method of treating a subject afflicted with a proliferative disorder comprising administering to the subject a therapeutically effective amount of the composition of claim 1.

33.-40. (canceled)


SEQUENCE LISTING (12 sequences)

SEQ ID NO: 1 - RNA, 5983 nt, Homo_sapiens
  ref <308>: DATABASE ACCESSION NUMBER: NM_006039.3
  ref <309>: DATABASE ENTRY DATE: 2011-12-17
  ref <313>: RELEVANT RESIDUES IN SEQ ID NO: (1)..(5983)
gaaggcgaggcggggagagacugggaaacagagcagcaagagaaagggcgcacgcugggguccuccuggacccggcguguccuccucuccgccaccccuuucucucccucccgacgcucuagccccagacccgcgccuccuccccuuccccucccacgcugcccacuucccuuccccucuaccccucgaacccuccuccccgguucugcgcuccucccuccgcccucccuccgcccgggcugccuccucccuccuccuccccgggaggcaucacuucgucccgacccggaggaggacgcgagccccuugcgggcggucaucacagcccagccucggggcugccacagcgcguugcgccugugcgcccucgguccccgcguccacugagcgccgcgcucggggauggggcccggccggccggcccccgcgcccuggccucgucaccugcugcgcugcguccugcuccucgggugccugcaccucggccgucccggcgccccuggggacgccgcccucccggaacccaacgucuuccucaucuucagccauggacugcagggcugccuggaggcccagggcgggcaggucagagucaccccggcuugcaauaccagccucccugcccagcgcuggaagugggucucccgaaaccggcuauucaaccuggguaccaugcagugccugggcacaggcuggccaggcaccaacaccacggccucccugggcauguaugagugugaccgggaagcacugaaucuucgcuggcauugucguacacugggugaccagcuguccuugcuccugggggcccgcaccagcaacauauccaagccuggcacccuugagcguggugaccagacccgcaguggccaguggcgcaucuacggcagcgaggaggaccuaugugcucugcccuaccacgaggucuacaccauccagggaaacucccacggaaagccgugcaccauccccuucaaauaugacaaccagugguuccacggcugcaccagcacgggccgcgaggauggucaccuguggugugccaccacccaggacuacggcaaagacgagcgcuggggcuucugccccaucaagaguaacgacugcgagaccuucugggacaaggaccagcugacugacagcugcuaccaguuuaacuuccaguccacgcugucguggagggaggccugggccagcugcgagcagcagggugcggaucugcugagcaucacggagauccacgagcagaccuacaucaacggccuccucacuggguacagcuccacccuguggaucggcuugaaugacuuggacacgagcggaggcuggcaguggucggacaacucgccccucaaguaccucaacugggagagugaccagccggacaaccccagugaggagaacuguggagugauccgcacugaguccucgggcggcuggcagaaccgugacugcagcaucgcgcugcccuaugugugcaagaagaagcccaacgccacggccgagcccaccccuccagacaggugggccaaugugaagguggagugcgagccgagcuggcagcccuuccagggccacugcuaccgccugcaggccgagaagcgcagcuggcaggaguccaagaaggcaugucuacggggcgguggcgaccuggucagcauccacagcauggcggagcuggaauucaucaccaagcagaucaagcaagagguggaggagcuguggaucggccucaacgauuugaaacugcagaugaauuuugaguggucugacgggagccuugugagcuucacccacuggcaccccuuugagcccaacaacuuccgggacagucuggaggacugugucaccaucuggggcccggaaggccgcuggaacgacagucccuguaaccaguccuugccauccaucugcaagaaggcaggccagcugagccagggggccgccgaggaggaccauggcugccggaaggguuggacguggcacagcccauccugcuacuggcugggagaagaccaagugaccuacagugaggcccggcgccugugcacugaccauggcucucagcuggucaccaucaccaacagguucgagcaggccuucgucagcagccucaucuacaacugggagggcgaguacuucuggacggcccugcaggaccucaacagcaccggcuccuucuucuggcucaguggggaugaagucauguacacccacuggaaccgggaccagcccggguacagccgugggggcugcguggcgcuggccacuggcagcgccauggggcugugggaggugaagaacuguaccucguuccgggcccgcuacaucugccggcagagccugggcacuccagugacgccggagcugccggggccagaucccacgcccagccucacuggcuccuguccccagggcugggccucggacaccaaacuccgguauugcuauaagguguucagcucagagcggcugcaggacaagaagagcuggguccaggcccagggggccugccaggagcugggggcccagcugcugagccuggccagcuacgaggaggagcacuuuguggccaacaugcucaacaagaucuucggugaaucagaacccgagauccacgagcagcacugguucuggaucggccugaaccgucgggaucccagagggggucagaguuggcgcuggagcgacggcguaggguucucuuaccacaauuucgaccggagccggcacgacgacgacgacauccgaggcugugcggugcuggaccuggccucccugcaguggguggccaugcagugcgacacacagcuggacuggaucugcaagauccccagagguacggacgugcgggagcccgacgacagcccucaaggccgacgggaauggcugcgcuuccaggaggccgaguacaaguucuuugagcaccacuccacgugggcgcaggcgcagcgcaucugcacgugguuccaggccgagcugaccucggugcacagccaggcggagcuagacuuccugagccacaacuugcagaaguucucccgggcccaggagcagcacugguggaucggccugcacaccucugagagcgaugggcgcuucagauggacagaugguuccauuauaaacuucaucuccugggcaccaggcaaaccucggccugucggcaaggacaagaagugcguguacaugacagccagccgagaggacuggggggaccagaggugccugacagccuugcccuacaucugcaagcgcagcaacgucaccaaagaaacgcagcccccagaccugccaacuacagcccuggggggcugccccucugacuggauccaguuccucaacaaguguuuucagguccagggccaggaaccccagagccgggugaaguggucagaggcacaguucuccugugaacagcaagaggcccagcuggucaccaucacaaaccccuuagagcaagcauucaucacagccagccugcccaaugugaccuuugaccuuuggauuggccuccaugccucgcagagggacuuccaguggguggagcaggagccuuugauguaugccaacugggcaccuggggagcccucuggcccuagcccugcucccaguggcaacaaaccgaccagcugugcagugguccugcacagccccucagcccacuucacuggccgcugggacgaucggagcugcacggaggagacccauggcuucaucugccagaagggcacggaccccucccugagcccguccccagcagcgcugccccccgccccgggcacugagcucuccuaccucaacggcaccuuccggcugcuucagaagccgcugcgcuggcacgaugcccuccugcugugugagagccgcaaugccagccuggccuacgugcccgaccccuacacccaggccuuccucacgcaggcugcccgagggcugcgcacgccgcucuggauugggcuggcuggcgaggagggcucucggcgguacuccugggucucagaggagccgcugaacuacgugggcuggcaggacggggagccgcagcagccggggggcuguaccuacguagauguggacggggccuggcgcaccaccagcugugacaccaagcugcagggggcugugugugggguuagcagugggcccccuccuccccgaagaauaagcuaccauggcagcuguccccagggacuggcagacuccgcguggauucccuuccgggagcacugcuauucuuuccacauggagcugcugcugggccacaaggaggcgcgacagcgcugccagagagcggguggggccguccugucuauccuggaugagauggagaauguguuugucugggagcaccugcagagcuaugagggccagagucggggcgccuggcugggcaugaacuucaaccccaaaggaggcacucuggucuggcaggacaacacagcugugaacuacuccaacugggggcccccgggcuugggccccagcaugcugagccacaacagcugcuacuggauucagagcaacagcgggcuauggcgccccggcgcuugcaccaacaucaccaugggugucgucugcaagcuuccucgugcugagcagagcagcuucuccccaucagcgcuuccagagaacccagcggcccuggugguggugcugauggcggugcugcugcuccuggccuugcugaccgcagcccucauccuuuaccggaggcgccagagcaucgagcgcggggccuuugagggugcccgcuacagccgcagcagcuccagccccaccgaggccacugagaagaacauccuggugucagacauggaaaugaaugagcaacaagaauagagccaggcgcgugggcagggccagggcgggaggagcuggggagcuggggcccugggucagucuggccccccaccagcugccuguccaguuggccuauggaagggugcccuugggagucgcuguugggagccggagcugggcagagccugggcugguggggugccacccucccacaagggcugggcugagacccagcugagugcagcguggcguuucccuuucugggggggccugaggucuugucaccugguccugugcccccacaggaaccagagguaggaugggagggggaacgagagccucuuucuccccagagcccccggcccaggccuguugauccgcgccccaggacccccuucuuugcagagcccgaggagccuccccuguccccucgggcagaucuguugugucucucuucccaccuggcagccucagcucugugccccucacccugcucccucucgccccuucucucccaccccuuccuucugagccgggcccuggggauuggggagcccucuuguuccugaugagggucagcugagggggcugagcauccaucacuccugugccugcugggguggcuguggggcguggcaggaggggccuagguggguugggccugagaaccagggcacggguguggugucugcugggcuggagauaagacuggggagagacaccccaaccucccagggugggagcugggccgggcugggaugucaucuccugccgggcgggggagggcucugccccuggaagaguccccuguggggaccaaaauaaguucccuaacaucuccagcuccuggcucugguuuggagcaaggggaaggguugccagaguccugggggccccagaggagcaggagucugggagggcccagaguucacccucuaguggauccaggaggagcagcacccgagcccuggaguggcccaguacccuuccaagaggccacagucccagccaggacaaaguaugcggcccauccuggugcgacagcgugggacaaugugaacauggacucgaagacauggcccuuucucuguaguugauuuuuuaaaugugccauuauuguuuuuaaaaaaaaaggaaaaaagaaaagcaaacaaauaaaacaccuuuaagaggcuugaaa

SEQ ID NO: 2 - protein, 1479 aa, Homo_sapiens
  ref <308>: DATABASE ACCESSION NUMBER: NP_006030.2
  ref <309>: DATABASE ENTRY DATE: 2012-11-17
  ref <313>: RELEVANT RESIDUES IN SEQ ID NO: (1)..(1479)
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKLPRAEQSSFSPSALPENPAALVVVLMAVLLLLALLTAALILYRRRQSIERGAFEGARYSRSSSSPTEATEKNILVSDMEMNEQQE

SEQ ID NO: 3 - DNA, 1593 bp, ARTIFICIAL
  Extracellular domain of human ENDO180 (amino acids 1-522) cloned within FLAG sequence
atggggcccggccggccggcccccgcgccctggcctcgtcacctgctgcgctgcgtcctgctcctcgggtgcctgcacctcggccgtcccggcgcccctggggacgccgccctcccggaacccaacgtcttcctcatcttcagccatggactgcagggctgcctggaggcccagggcgggcaggtcagagtcaccccggcttgcaataccagcctccctgcccagcgctggaagtgggtctcccgaaaccggctattcaacctgggtaccatgcagtgcctgggcacaggctggccaggcaccaacaccacggcctccctgggcatgtatgagtgtgaccgggaagcactgaatcttcgctggcattgtcgtacactgggtgaccagctgtccttgctcctgggggcccgcaccagcaacatatccaagcctggcacccttgagcgtggtgaccagacccgcagtggccagtggcgcatctacggcagcgaggaggacctatgtgctctgccctaccacgaggtctacaccatccagggaaactcccacggaaagccgtgcaccatccccttcaaatatgacaaccagtggttccacggctgcaccagcacgggccgcgaggatggtcacctgtggtgtgccaccacccaggactacggcaaagacgagcgctggggcttctgccccatcaagagtaacgactgcgagaccttctgggacaaggaccagctgactgacagctgctaccagtttaacttccagtccacgctgtcgtggagggaggcctgggccagctgcgagcagcagggtgcggatctgctgagcatcacggagatccacgagcagacctacatcaacggcctcctcactgggtacagctccaccctgtggatcggcttgaatgacttggacacgagcggaggctggcagtggtcggacaactcgcccctcaagtacctcaactgggagagtgaccagccggacaaccccagtgaggagaactgtggagtgatccgcactgagtcctcgggcggctggcagaaccgtgactgcagcatcgcgctgccctatgtgtgcaagaagaagcccaacgccacggccgagcccacccctccagacaggtgggccaatgtgaaggtggagtgcgagccgagctggcagcccttccagggccactgctaccgcctgcaggccgagaagcgcagctggcaggagtccaagaaggcatgtctacggggcggtggcgacctggtcagcatccacagcatggcggagctggaattcatcaccaagcagatcaagcaagaggtggaggagctgtggatcggcctcaacgatttgaaactgcagatgaattttgagtggtctgacgggagccttgtgagcttcacccactggcacccctttgagcccaacaacttccgggacagtctggaggactgtgtcaccatctggggcccggaaggccgctggaacgacagtccctgtaaccagtccttgccatccatctgcaagaaggcaggccagctgagccagggggccgccgaggaggaccatggctgccgggattacaaggacgacgacgataagtga

SEQ ID NO: 4 - protein, 530 aa, ARTIFICIAL
  Polypeptide encoded by SEQ ID NO:3
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRDYKDDDDK

SEQ ID NO: 5 - DNA, 1127 bp, ARTIFICIAL
  Polynucleotide seq of anti-ENDO180 scFv clone G7V
taatgtgagttagctactcttaggcaccccaggctttacactttatgcttccggctcgtatgttgtgtggaattgtgagcggataacaatttcacacaggaaacagctatgaccatgattacgccaagcttgccaaattctatttcaaggagacagtcataatgaaatacctattgcctacggcagccgctggattgttattactcgcagcaagcggcgcgcatgcccaggtgcagctggtgcagtctgggggaggcctggtcaagcctggggggtccctgagactctcctgtgcagcctctggattcaccttcagtagctatagcatgaactgggtccgccaggctccagggaaggggctggagtgggtggccaacataaagccagatggaagtgagagacactctgtggactctgtgaagggccgattcaccatctccagagacaactccaagaactcactgtatctgcaaatgaacagcctgagagccgaggacacggctgtttattactgtgcgcgacccggggctgggcgacttgactactggggccagggcaccctggtcaccgtctcctcaggtggaggcggttcaggcggaggtggctctggcggtggcggatcgaattttatgctgactcaggacgctgctgtgtctgtggccttgggacagacagtcaggatcacatgccaaggagacagcctcagaagctattatgcaagctggtaccaacagaagccaggacaggcccctgtacttgtcgtctatggtaaaaacaaccgaccctcagggatcccagaccgattctctggctccagctcaggaaacacagcttccttgaccatcactggggctcaggcggaagatgaggctgactattactgtaactcccgggacagcagtggtaacccctgggcgttcggcggagggaccaaggtgaccgtcctagctagcggcaaaccaatcccaaacccactgctgggcctggatagtactcaccatcaccatcaccattaggcggccgctactgttgaaagttgtttagcaaaacctcatacagaaaattcatttactaacgtctggaaagacgacaaaactttagatcgttacgctaactatgagggcttg

SEQ ID NO: 6 - protein, 284 aa, ARTIFICIAL
  Polypeptide encoded by SEQ ID NO:5
MKYLLPTAAAGLLLLAASGAHAQVQLVQSGGGLVKPGGSLRLSCAASGFTFSSYSMNWVRQAPGKGLEWVANIKPDGSERHSVDSVKGRFTISRDNSKNSLYLQMNSLRAEDTAVYYCARPGAGRLDYWGQGTLVTVSSGGGGSGGGGSGGGGSNFMLTQDAAVSVALGQTVRITCQGDSLRSYYASWYQQKPGQAPVLVVYGKNNRPSGIPDRFSGSSSGNTASLTITGAQAEDEADYYCNSRDSSGNPWAFGGGTKVTVLASGKPIPNPLLGLDSTHHHHHH

SEQ ID NO: 7 - protein, 12 aa, ARTIFICIAL
  CDR domain
CARPGAGRLDYW

SEQ ID NO: 8 - protein, 13 aa, ARTIFICIAL [sequence omitted]

SEQ ID NO: 9 - RNA, 19 nt, ARTIFICIAL
  Chemically synthesized
gccacaacgucuauaucau

SEQ ID NO: 10 - RNA, 19 nt, ARTIFICIAL
  Chemically synthesized
augauauagacguuguggc

SEQ ID NO: 11 - RNA, 18 nt, ARTIFICIAL
  Chemically synthesized
gugcaaagugguauccua

SEQ ID NO: 12 - RNA, 19 nt, ARTIFICIAL
  Chemically synthesized
uaggauaccacuuugcacg